(19) United States
(12) Patent Application Publication
(10) Pub. No.: US 2019/0006026 A1
(43) Pub. Date: Jan. 3, 2019

(54) MEDICAL INFORMATION ANALYSIS SYSTEMS AND METHODS

(71) Applicant: Drug Development Analytics LLC, Summit, NJ (US)

(72) Inventor: Margaret E. Hurley, Summit, NJ (US)

(21) Appl. No.: 16/126,749

(22) Filed: Sep. 10, 2018

Related U.S. Application Data
(63) Continuation of application No. 14/706,334, filed on May 7, 2015.
(60) Provisional application No. 61/989,943, filed on May 7, 2014.

(51) Int. Cl. G16H 10/20; G06Q 50/22; G06Q 10/06

(52) U.S. Cl. CPC G16H 10/20; G06Q 50/22; G06Q 10/063; G06F 19/36

(57) ABSTRACT

A method for evaluating drug study information to determine suitability of the drug study information for acceptance by a regulatory authority. The method includes receiving a drug study identification and study type, indicating whether the drug study is either (i) a nonclinical study that is intended to be a Good Laboratory Practice study, or (ii) a clinical study that is intended to support the efficacy indication of a drug that is the subject of the drug study information, and associating one or more checklists with the drug study. Each checklist has elements identifying one or more features to be identified in the drug study information, and a query to select a deficiency type from a predetermined list of deficiency types. The method receives the deficiency types and automatically assigns a criticality value to each based on the nature of the type of drug study.

This application is a continuation of U.S. application Ser. No. 14/706,334 filed on May 7, 2015 which claims the benefit of U.S. Provisional Application No. 61/989,943, filed May 7, 2014, the entire contents of which are incorporated herein by reference.

### FIELD OF THE INVENTION

This application relates to electronic systems and associated methods for assessing information relating to experimental drugs or the like, to provide indications of the suitability of the information to support regulatory approval or other useful information.

### BACKGROUND OF THE INVENTION

The process of developing a new drug, medical treatment or device typically takes many years, and costs many millions of dollars. In many cases, particularly for new drugs, a regulatory body must approve the drug before it is allowed to be sold on the market. The regulatory body typically requires the drug to pass a number of tests, and otherwise be proven to be safe and efficacious for treatment of one or more conditions. Examples of regulatory bodies that oversee the introduction of new drugs include the United States Food and Drug Administration (“FDA”), the European Medicines Agency (“EMEA”), Health Canada, and so on. Furthermore, bodies such as the International Conference on Harmonisation of Technical Requirements for Registration of Pharmaceuticals for Human Use (“ICH”) may promote additional guidelines for new drug testing or approval. Each regulatory body may have its own requirements to obtain approval of any given drug.

A large part of the cost of obtaining approval typically lies in the testing that is performed to satisfy the regulatory body's approval requirements. A single study can cost millions of dollars and take years to complete. Such tests may be in vitro and/or in vivo, and can involve a number of different phases of clinical trials. Even where a drug is already known and approved, the regulatory body may require further testing or approval procedures to permit alternative formulations, uses or delivery methods of the drug, or to allow entities other than the original sponsor to market the drug.

As a consequence of the many regulatory requirements and the great complexity of the supporting information (i.e., the testing and other data that are required to support approval), the drug approval process has become notorious for being a very difficult, expensive, and time-consuming process. Furthermore, interpretations of the regulations and supporting information can vary, resulting in frequent disputes between the drug sponsor and the FDA or other regulatory body reviewing the drug or other new product. Thus, drug sponsors typically make some effort to evaluate the completeness of the supporting information before submitting it to the regulatory body, to help ensure that all of the relevant requirements are satisfied. Identifying errors or missing information before submitting the drug application can safe a significant amount of time, and may reduce the cost of any additional tests that may be required.

In the past, electronic systems have been used to attempt to track information provided in support of a drug approval effort. For example simple spreadsheets and databases of checklists have been used to track whether drug study information conforms with various regulatory requirements. For example, checklists in spreadsheet form have been used to track whether the supporting information satisfies or does not satisfy certain regulatory criteria. However, such systems and procedures could be cumbersome to use, and generally required exceptional user skill to ensure that all of the appropriate checklists were considered (and inappropriate checklists were not considered). Such systems also merely indicated whether a particular aspect of the supporting information was defective, and were unable to further analyze this output to provide a realistic assessment as to whether the defect could or would affect the likelihood of an application being accepted. Thus, such systems were unreliable to the extent that persons reviewing the study information could make erroneous subjective determinations of which checklists should be used, and whether any deficiencies were sufficient to jeopardize the filing. Such systems also were unable to provide input about how to remedy any deficiencies that might be found. Such systems also were unable to provide real-time access to instructions relevant to answer the many questions that might be asked of a new drug or product, and were not configured to help train and improve the user, or to help ensure consistent interpretation of the supporting information and assessment of the new drug application. Such systems also had other deficiencies, as will be appreciated by persons of ordinary skill in the art.

### SUMMARY OF THE INVENTION

The inventions are described herein by reference to exemplary embodiments. These embodiments are intended to address various problems with prior methods and systems for seeking to obtain regulatory approval for drugs, medical devices, and the like. However, the invention is not limited to solving any or all of the deficiencies described herein, and it will be appreciated that other advantages and uses of the inventions will be appreciated through practice of the invention.

In one aspect, there is provided a computer-implemented method for evaluating drug study information to determine suitability of the drug study information for acceptance by a regulatory authority. The method includes executing with a processor the steps of: receiving, from at least one user interface, an identification of a drug study associated with the drug study information; receiving, from the at least one user interface, a selection of a study type for the drug study, the study type being selected from a plurality of predetermined study types maintained in a memory accessible by the processor; receiving, from the at least one user interface, an indication whether the drug study is either (i) a nonclinical study that is intended to be a Good Laboratory Practice study, or (ii) a clinical study that is intended to support the efficacy indication of a drug that is the subject of the drug study information; and associating at least one checklist with the drug study, the at least one checklist being selected from a plurality of predetermined checklists maintained in the memory based on the identity of the selected study type. The at least one checklist includes a plurality of elements, each element having a respective identification of one or more features to be identified in the drug study information, and a respective one or more queries associated with the respective one or more features to be identified in the drug study information. The one or more queries include a query to select a deficiency type from a predetermined list of deficiency types. The method also includes the steps of: presenting the plurality of elements to the at least one user interface; receiving, from the at least one user interface, a respective reply to each of the plurality of elements; and assigning, by the processor, a respective criticality value to each respective reply comprising a selection of a respective deficiency type from the predetermined list of deficiency types. The step of assigning a criticality value includes assigning a high respective criticality value if the drug study is either (i) a nonclinical study that is intended to be a Good Laboratory Practice study, or (ii) a clinical study that is intended to support the efficacy indication of a drug that is the subject of the drug study information, or assigning a moderate respective criticality value if the drug study is not (i) a nonclinical study that is intended to be a Good Laboratory Practice study, and not (ii) a clinical study that is intended to support the efficacy indication of a drug that is the subject of the drug study information.

A second aspect may modify the first aspect by further including the step of adding one or more additional elements to the plurality of elements if the drug study is either (i) a nonclinical study that is intended to be a Good Laboratory Practice study, or (ii) a clinical study that is intended to support the efficacy indication of a drug that is the subject of the drug study information.

A third aspect may modify the second aspect by providing the one or more additional elements as one or more additional features to be identified in the drug study information. The one or more additional features may be aspects of (i) a bioanalytical method validation report, (ii) an analytical method validation report, or (iii) a biological sample analysis report.

A fourth aspect may modify the second or third aspect by having the step of associating at least one additional checklist with drug study be performed automatically by the processor.

A fifth aspect may modify the second or third aspect by having the step of associating at least one additional checklist with drug study be performed by receiving a selection of the at least one additional checklist from the at least one user interface.

A sixth aspect may modify any of the previous aspects by having the predetermined list of deficiency types include a high risk group of deficiency types representing deficiencies that affect the acceptability of the drug study information and a low risk group of deficiency types representing deficiencies that do not affect the acceptability of the drug study information.

A seventh aspect may modify the sixth aspect by having the step of assigning, by the processor, a respective criticality value to each respective reply comprising a selection of a respective deficiency type from the predetermined list of deficiency types further comprise assigning a low respective criticality value if the respective reply comprises a respective deficiency type selected from the low risk group of deficiency types, regardless of whether the drug study is either (i) a nonclinical study that is intended to be a Good Laboratory Practice study, or (ii) a clinical study that is intended to support the efficacy indication of a drug that is the subject of the drug study information.

An eighth aspect may modify any of the preceding aspects by having at least one of the plurality of elements include a trigger element having a question regarding the content of the drug study information, and an associated dynamic checklist having one or more additional elements regarding the drug study information; and having the method further include adding the one or more additional elements to the plurality of elements upon receiving, from the user interface, a predetermined reply to the question regarding the content of the drug study information.

A ninth aspect may modify the eighth aspect by having the question regarding the content of the drug study information be a question whether the drug study information includes kinetic or blood level data.

A tenth aspect may modify the eighth aspect by having the question regarding the content of the drug study information be a question whether the drug study information includes metabolism and transporter drug interaction-related data.

An eleventh aspect may modify the eighth aspect by having the question regarding the content of the drug study information be a question whether the drug study information includes toxicology-related data.

A twelfth aspect may modify any of the eighth through eleventh aspects by having the step of adding the one or more additional elements to the plurality of elements be performed by the processor automatically associating one or more additional checklists to the drug study.

A thirteenth aspect may modify any of the eighth through eleventh aspects by having the step of adding the one or more additional elements to the plurality of elements be performed by: presenting a list of one or more additional checklists to the at least one user terminal; receiving a selection of at least one of the one or more additional checklists from the at least one user terminal; and associating the selected at least one additional checklist with the drug study.

A fourteenth aspect may modify the thirteenth aspect by having the step of presenting a list of one or more additional checklists to the at least one user terminal include presenting a list of additional drug studies to which the one or more additional checklists were previously associated.

A fifteenth aspect may modify the thirteenth or fourteenth aspect by further including the steps of receiving a selection of one or more additional drug studies to which to add at least one of the one or more additional checklists; and associating the selected at least one additional checklist with the one or more additional drug studies.

A sixteenth aspect may modify any of the preceding aspects by having the one or more queries further include: a review status indicator to indicate whether the drug study information has been evaluated to respond to the respective element, an applicability indicator to indicate whether the element is applicable to the drug study information, and an acceptability indicator to indicate whether the drug study element contains the one or more features to the be identified in the drug study information.

A seventeenth aspect may modify any of the preceding aspects by having at least one of the elements include a regulatory source for information regarding the one or more features to be identified in the drug study information.

An eighteenth aspect may modify the seventeenth aspect by further including the step of presenting, to the at least one user interface, a hyperlink to the regulatory source.

A nineteenth aspect may modify any of the preceding aspects by having at least one of the deficiency types from the predetermined list of deficiency types include an associated record indicating a corrective action to take to overcome the deficiency type.

A twentieth aspect may modify any of the preceding aspects by further including the step of compiling a list of the respective criticality values for each of the one or more checklists associated with the drug study, and indicating the likelihood that the drug study information will be accepted by the regulatory authority.

A twenty-first aspect may modify any of the preceding aspects by having at least one of the one or more queries for at least one of the plurality of elements include the question of whether the drug study is either (i) a nonclinical study that is intended to be a Good Laboratory Practice study, or (ii) a clinical study that is intended to support the efficacy indication of a drug that is the subject of the drug study information.

A twenty-second aspect may modify any of the preceding aspects by repeating the steps of presenting the elements to the user interface, receiving replies for each element, and assigning a criticality value to each reply that includes a deficiency type selected from the list of predetermined deficiency types.

A twenty-third aspect may modify the twenty-second aspect by further including the step of identifying any differences in the respective criticality value of the first reply and the respective criticality value of each subsequent reply to each element.

A twenty-fourth aspect may modify the twenty-third aspect by further including the step of resolving the differences and providing a final respective criticality value of each respective reply.

A twenty-fifth aspect may modify any of the preceding aspects by further including the steps of (a) presenting, the plurality of elements to the at least one user interface during multiple successive review stages, each review stage being based on a respective modified version of the drug study information; (b) receiving, from the at least one user interface, a respective reply to each of the plurality of elements during each of the multiple successive review stages; (c) assigning, by the processor, during each of the multiple successive review stages, a respective criticality value to each respective reply comprising a selection of a respective deficiency type from the predetermined list of deficiency types by: assigning a high respective criticality value if the drug study is either (i) a nonclinical study that is intended to be a Good Laboratory Practice study, or (ii) a clinical study that is intended to support the efficacy indication of a drug that is the subject of the drug study information, or assigning a moderate respective criticality value if the drug study is not (i) a nonclinical study that is intended to be a Good Laboratory Practice study, and not (ii) a clinical study that is intended to support the efficacy indication of a drug that is the subject of the drug study information; and repeating steps (a)-(c) until none of the respective replies is assigned a high respective criticality value.

A twenty-sixth aspect may modify any of the preceding aspects by having the step of associating at least one checklist with the drug study be performed automatically by the processor.

A twenty-seventh aspect may modify any of the preceding aspects by having the step of associating at least one checklist with the drug study be performed by receiving an association from the at least one user interface.

Other aspects of the invention will be apparent from the disclosures provided herein.

### BRIEF DESCRIPTION OF THE DRAWINGS

Exemplary embodiments are described herein by reference to the attached drawings. The attached drawings illustrate examples of embodiments encompassed within the scope of the present disclosure, and, therefore, are not to be considered limiting. To facilitate understanding, like reference numerals have been used, where possible, to designate like elements common to the figures.

FIG. 1 depicts a block diagram of an exemplary system in accordance with one embodiment of the present disclosure.

FIG. 2 is an embodiment of a login prompt that may be used with exemplary embodiments of the invention.

FIG. 3 is an embodiment of a module selection interface that may be used with exemplary embodiments of the invention.

FIG. 4 is an embodiment of an application menu interface that may be used with exemplary embodiments of the invention.

FIG. 5 is a flowchart illustrating a project setup process that may be used with exemplary embodiments of the invention.

FIG. 6A is an embodiment of a study entry interface that may be used with exemplary embodiments of the invention.

FIG. 6B is an embodiment of another study entry interface that may be used with exemplary embodiments of the invention.

FIG. 7 is an embodiment of an exemplary list of study types that may be used with exemplary embodiments of the invention.

FIG. 8 is an embodiment of a study type generation interface that may be used with exemplary embodiments of the invention.

FIG. 9 is an embodiment of an exemplary list of checklists that may be used with exemplary embodiments of the invention.

FIG. 10 is an embodiment of a checklist element interface that may be used with exemplary embodiments of the invention.

FIG. 11 is an embodiment of a checklist element viewing or editing interface that may be used with exemplary embodiments of the invention.

FIG. 12 is an embodiment of a study list interface that may be used with exemplary embodiments of the invention.

FIG. 13 is an embodiment of a study overview interface that may be used with exemplary embodiments of the invention.

FIG. 14 is an embodiment of a task prompt that may be used with exemplary embodiments of the invention.

FIG. 15 is an embodiment of a checklist overview interface that may be used with exemplary embodiments of the invention.

FIG. 16 is an embodiment of another checklist overview interface that may be used with exemplary embodiments of the invention.

FIG. 17 is an embodiment of a checklist addition prompt that may be used with exemplary embodiments of the invention.

FIG. 18 is an embodiment of an element speed entry prompt that may be used with exemplary embodiments of the invention.

FIG. 19 is an embodiment of an extended element entry prompt that may be used with exemplary embodiments of the invention.

FIG. 20 is an embodiment of another extended element entry prompt that may be used with exemplary embodiments of the invention.

FIG. 21 is an embodiment of a corrections task interface that may be used with exemplary embodiments of the invention.

FIG. 22 is an embodiment of a program level guidance interface that may be used with exemplary embodiments of the invention.

FIG. 23 is an embodiment of a deficiency summary report that may be used with exemplary embodiments of the invention.

FIG. 24 is an embodiment of another deficiency summary report that may be used with exemplary embodiments of the invention.

FIG. 25 is an embodiment of a checklist redline report that may be used with exemplary embodiments of the invention.

FIG. 26 is an embodiment of an audit report that may be used with exemplary embodiments of the invention.

FIG. 27 is an embodiment of an audit detail interface that may be used with exemplary embodiments of the invention.

FIG. 28 is a flowchart illustrating a process flow and data interactions that may be used with exemplary embodiments of the invention.

### DETAILED DESCRIPTION OF EXEMPLARY EMBODIMENTS

The present disclosure provides examples of a number of inventions that, for convenience, are described collectively in the context of a system for evaluating information relating to drug and medical device regulatory approval. It will be appreciated that the various inventions described herein may be used separately, or in various combinations, and the scope of the claimed invention is not intended to be limited to the collective features and functions of the systems described herein.

The described embodiments generally relate to systems and methods for evaluating documentation (including its content and format) to determine whether the documentation is sufficient and appropriate to satisfy relevant regulatory requirements, such as regulatory requirements associated with applications for approval of drugs or other medical devices or products. The system may be configured for use in preparing and reviewing regulatory filings for drugs, products or devices, and may be used at various stages of the approval process. For example, the system may be configured to support Investigational New Drug Applications (“INDs”), Investigational Device Exemptions (“IDEs”), 510(k) filings, New Drug Applications (“NDAs”), Abbreviated New Drug Applications (“ANDAs”), assessments at intermediate stages of drug development, and submissions of completed applications to health authorities for marketing approval.

More specifically, the embodiments described herein relate to systems and methods for evaluating studies and other information (collectively, “supporting information”) that will be provided to a Health Authority to support the marketing approval of new drugs, devices, and/or the like or the request to study the proposed product that is being developed for commercialization. At its most basic level, the system can be used, in one configuration, to analyze the information to determine whether it meets the regulatory requirements, and automatically characterize any deficiencies or potential approval concerns that might be found. The system may be configured to address requirements that are specific to one or more health authorities, such as the FDA, EMEA, Health Canada, etc. In performing this function, the systems and methods may be used to effectively prescreen applications for potential approval by a regulatory authority, and identify any issues that are likely to cause or that may cause a denial of approval. The system also may generate documents that identify which requirements are not met, and provide recommended corrective actions.

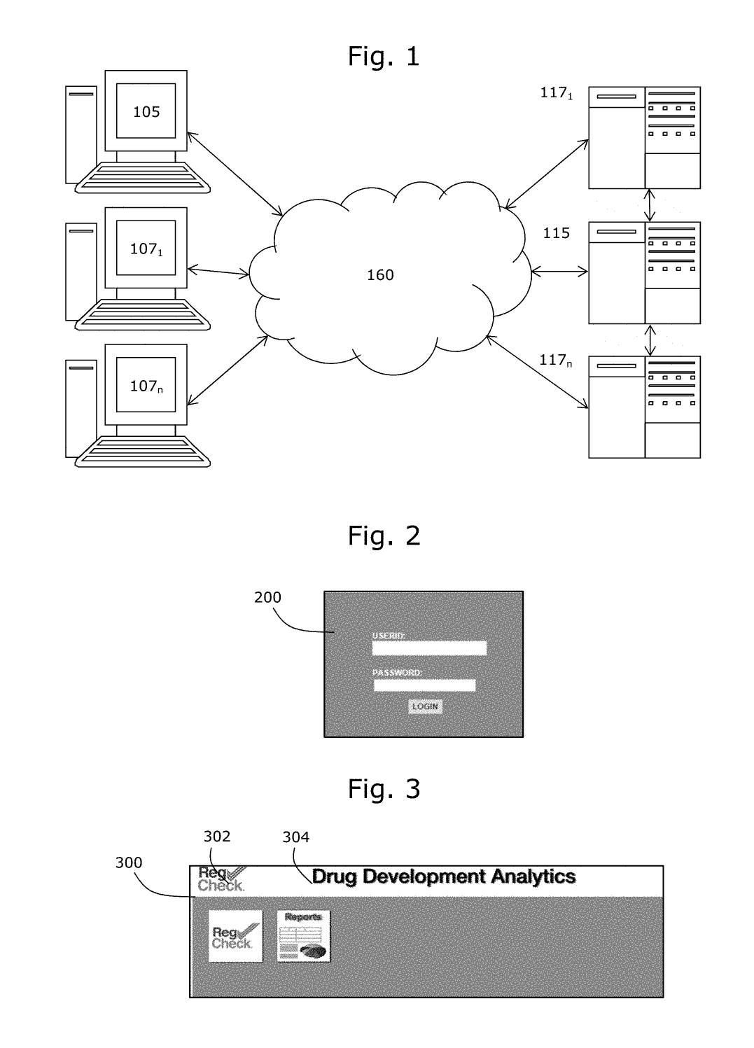

FIG. 1 is a system-level network diagram illustrating an exemplary technical platform for embodiments of the invention. The system 100 preferably operates on a global computer/communication network. However, some or all aspects of the system 100 may be self-contained within a single computer to allow operation “offline.” The system 100 generally comprises clients, shown as a first client 105 and one or more secondary clients 107₁ through 107ₙ (“n” representing any number of additional clients), each in communication with an administrator, which may be hosted on a central server 115, and optionally one or more secondary servers 117₁-117n. Communication, in this example, takes place through a network 160.

The clients and servers generally are provided in the form of computers that include specialized software or hardware configured to perform one or more of the exemplary methods described herein. As used herein, the term “computer” refers to any device that is capable of processing a signal or other information. Examples of computers include, without limitation, a personal computer, a portable computer, a handheld computer, a cellular phone, a smart phone, a digital tablet, a laptop computer, a netbook, a smart TV, a device configured to connect to the internet, an Internet appliance, a Personal Data Assistant (PDA), an application-specific integrated circuit (ASIC), a programmable logic array (PLA), a microcontroller, a digital logic controller, a digital signal processor (DSP), or the like, or may generally include a suitably configured processor and memory that are operatively associated to work together, as known in the art. Various input devices and displays may be used with the computer, also as known in the art. A computer also may include software in the form of programmable code, micro code, or firmware or other hardware embedded logic. A computer may include multiple processors that operate in parallel, and the processing performed by a computer may be distributed among multiple separate devices. The term computer encompasses all such devices and equivalents thereof, when configured to perform in accordance with the disclosed embodiments. Each of the client(s) and server(s) preferably is capable of transmitting data through the network 160 either in real time (i.e., where data is sent continuously back and forth), or in batched mode (i.e., where data is sent in bulk after an amount of work is performed).

The network 160 may comprise a global computer network (e.g., the Internet), a local area network, or any combination of networked computers or systems. The communications described herein can be accomplished using any kind of wired and/or wireless computing network or communications means capable of transmitting data or signals, such as a wireless and/or wired computing network allowing communication via, for example, an 802.11 wireless LAN (“WLAN”) protocols, “Wi-Fi” protocols, Bluetooth protocols, 60 Ghz Protocols (“WirlessHD” and “WiGig”), Wireless Home Automation protocols (“Z-Wave” and “Zigbee”), cellular data protocol (e.g., EDGE, CDMA, CDMA2000, TD-SCDMA, WCDMA, UMTS, HSDPA, EVDO, TDMA, FDMA, GSM, LTE, HSPA+, WiMAX, OFDMA), and/or the like. Suitable examples include a packet-switched network, a local area network (LAN), wide area network (WAN), virtual private network (VPN), or any other means of transferring data. The network 160 may be a partial or full deployment of most any communication/computer network or link, including any of, any multiple of, any combination of or any combination of multiples of a public or private, terrestrial wireless or satellite, and wireline networks or links. A single network 160 or multiple networks (not shown) that are communicatively coupled to one another can be used. It is contemplated that multiple networks of varying types can be connected together and utilized to facilitate the communications contemplated by the systems and elements described in this disclosure.

In one embodiment, the system is generally configured as a central server that is operated by an administrator to allow permissioned access by users that operate the clients. In this embodiment, the server 115 may be located at essentially any location, and may actually be embedded within a client computer (e.g., client 105 may also perform the functions of the server 115). The server 115 is configured to allow access by the clients through an accessible data portal, which communicates with each client through the network 160. The accessible data portal may comprise any number of security measures to provide a reasonably secure system, suitable for embodiments of the present invention. The accessible data portal may further comprise a graphical user interface (GUI) (e.g., a web-based portal) through which any of the first client 105 or secondary clients 107 may access the server 115.

The server 115 may also comprise a processor and operationally associated memory storing one or more databases or other sortable data storage memory to enable the system and methods disclosed herein. The database may be any commercially available storage database suitable for embodiments of the present disclosure, such as those systems commonly available under the names Oracle, DB2, Microsoft Access, Microsoft SQL, Postgres, MySQL, 4th Dimension, FileMaker, Alpha Five Database Management System, or the like. Often contained within the database is a plurality of data sets, each comprising specific data. A first data set may correlate to a first client 105, wherein a plurality of client-specific data is stored. The database may also include any number of subsequent data sets representing N clients, wherein N represents any number of clients practical for operation of embodiments of the present disclosure. The system may be configured to run more than one instance, multiple instances, and/or the like, of the exemplary systems and methods described herein.

The system users may include any person, business or entity, capable of participating in the system and methods disclosed herein. For example, the first client 105 may be a sponsor entity seeking regulatory approval for a drug, device, product, and/or the like (such a sponsor may act on its own behalf, or through an agent). The secondary clients 107 may include entities or persons that are providing services for the first client 105 in successfully navigating the regulatory process and analyzing data to ensure compliance with a regulatory authority. Alternatively, or in addition, the secondary clients 107 may be other sponsors that are unrelated to the first client 105. Furthermore, one or more of the first client 105 and the second clients 107 may be a regulatory authority, such as the FDA, that uses the system 100 during the approval process. In any of the foregoing configurations or other configurations, the administrator preferably controls access to the system by the clients, manages and maintains the operation of the system, and maintains the data in a secure environment.

The system 100 preferably includes secure access through a username and password combination or other security systems. An example of a login prompt 200 is shown in FIG. 2. The login prompt may comprise fields for entry of the user's user id and password, or the like. User accounts may be created by one or more users or entities connected to the system. Generally, each of the user accounts may correspond to one or more entities, whereby an entity may be an individual, a group, or other defined body, such as a drug sponsor, or the like. User registrations may be restricted, for example, to users selected by an administrator or the like. Certain data about a user may be verified to obtain a user ID and password. For example, the user's identity or active employment with a drug sponsor may need to be verified. Some embodiments may include alternative security measures, such as biometric security, key fob security token access, and/or the like. The system may provide multi-user login capabilities with different user access levels and the ability to manage user accounts and access levels.

After logging in, the user may be presented with a module selection interface 300, such as shown in FIG. 3, for accessing applications or modules associated with the system 100. For example, the user may be presented with the option of selecting either a regulatory and technical review module 302 or a reports module 304. The regulatory and/or technical review module may be configured to set up projects and to provide analysis of data received by the system to ensure the data meets requirements of a regulatory authority, for example, the FDA. The reports module may be configured to provide a user with a summary of all activity such as the deficiencies that need to be addressed for the potential approval process for a particular drug, or the like. The reports module may also be configured to generate customized reports after running an analysis on the data received by the system based on instructions and requirements received from a user and/or administrator. In some embodiments, the number and nature of the applications or modules presented to the user may depend on the user's login credentials. For example, the option to access the reports module 304 may only be provided to users with administrator-level credentials.

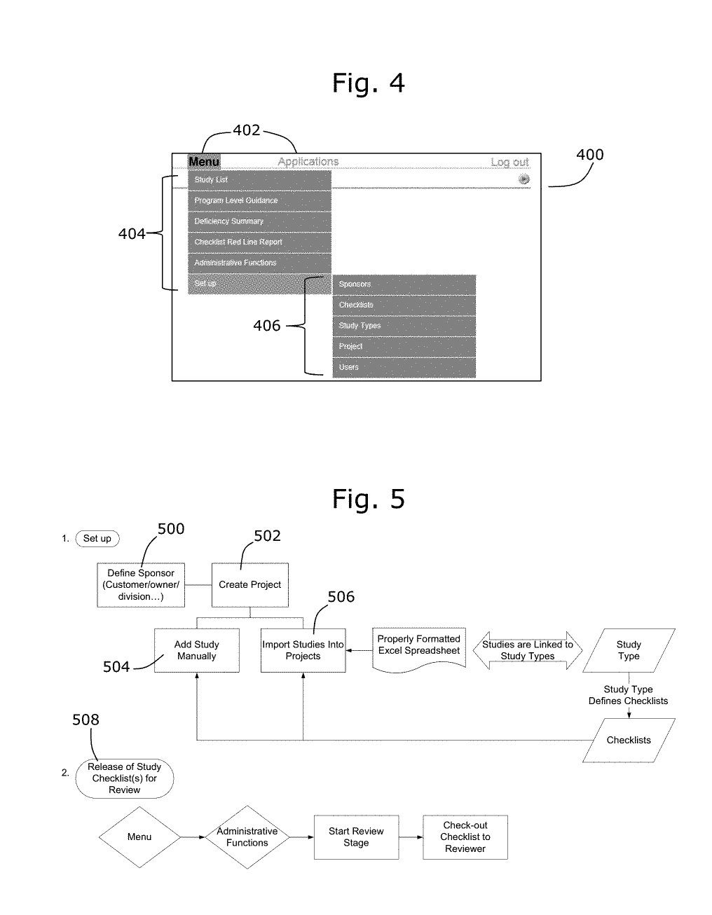

The system preferably is configured to have an administrative mode, and a user review mode. In general terms, the administrative mode is used to set up projects and perform final reviews, and the user review mode is used to perform the core information processing. An exemplary user interface 400 for the administrative mode is shown in FIG. 4. In this example, the interface is arranged as a series of tabs 402, which each may have an associated dropdown menu 404. One or more of the items in the dropdown menu 404 also may have an associated submenu 406. In this case, the “Menu” tab includes applications titled Study List, Program Level Guidance, Deficiency Summary, Checklist Red Line Report, Administrative Functions, and Set Up.

In this example, selecting the Set Up application opens the Set Up submenu 406, which includes applications titled Sponsors, Checklists, Study Types, Project, and Users. This submenu, which preferably is only displayed to users having administrator level or greater credentials, is used to set up sponsors, checklists, study types, projects and users.

The “sponsor” is the highest organizational level in the system. The sponsor designation can be used to identify an entity seeking approval of a drug or device. The sponsor may be identified in the system as a company, a division of a company, a consulting organization that works for one or more companies, and so on. Once a sponsor is created, one or more projects can be associated with each sponsor.

The projects are the second organizational level. One or more projects may be created for each drug that is being sponsored by the sponsor (multiple projects may be appropriate where the same drug is being evaluated for different indications of formulations). The administrator can create as many projects as desired for each sponsor.

Study types are the third organizational level. After creating a project, the administrator associates one or more study types with the project. Each study type typically designates a study typically conducted during drug development. As will become more apparent below, the purpose of assigning the study type is to ensure that the proper checklists (and their corresponding elements) are considered during review of the study. During the setup process, the administrator preferably adds all of the study types that are expected to be conducted for each project, based on the administrators assessment of the project details. The studies can address virtually any issue associated with the drug development process. For example, they may address quality issues, chemistry and manufacturing requirements, nonclinical study requirements (including but not limited to pharmacology, pharmacokinetics and metabolism studies), clinical study requirements such as efficacy, safety, and statistics, and formatting and electronic data set requirements.

The Set Up submenu 406 also allows the operator to set up users, such as administrators who have access to create projects and add new users, regular users (the lowest level of access) for users that perform project reviews, and final reviewers who have access to review the work of the regular users. The levels of access for each user can be tailored to specifically allow or disallow any number of system functions. The system preferably also includes the ability to block users, and track each user's system usage.

Finally, the Set Up menu includes an option to manually add checklists to studies. As explained in more detail below, checklists are lists that include various elements that are expected to be found in study information. Checklists (and elements of checklists) also may be included to reflect a sponsor's particular standard operating procedures (“SOPs”). For example a checklist may include questions asking whether certain reports include certain identification information that the sponsor requires in all of its documentation, such as tracking information, manufacturing information, and the like.

The system preferably includes one or more user interfaces to display all of the sponsors, projects, study types, checklists, and users that are entered into the system, and to which the particular user has access. Such an interface may include navigation features, such as word searching and sorting functions, to provide the administrator better access to the desired information.

FIG. 5 is a flowchart illustrating an exemplary project setup process. In step 500, the administrator creating or selects a sponsor. In step 502, the administrator creates or selects a project associated with the sponsor. Next, the administrator can proceed either to step 504 to manually add studies to the project from a list of available studies, or to step 506 to import studies into the project from a properly formatted source file, such as a spreadsheet in Excel format or the like. A hybrid process including both steps 504 and 506 is also possible. Regardless of how the studies are added to the project, it is preferred that each study has one or more associated checklists.

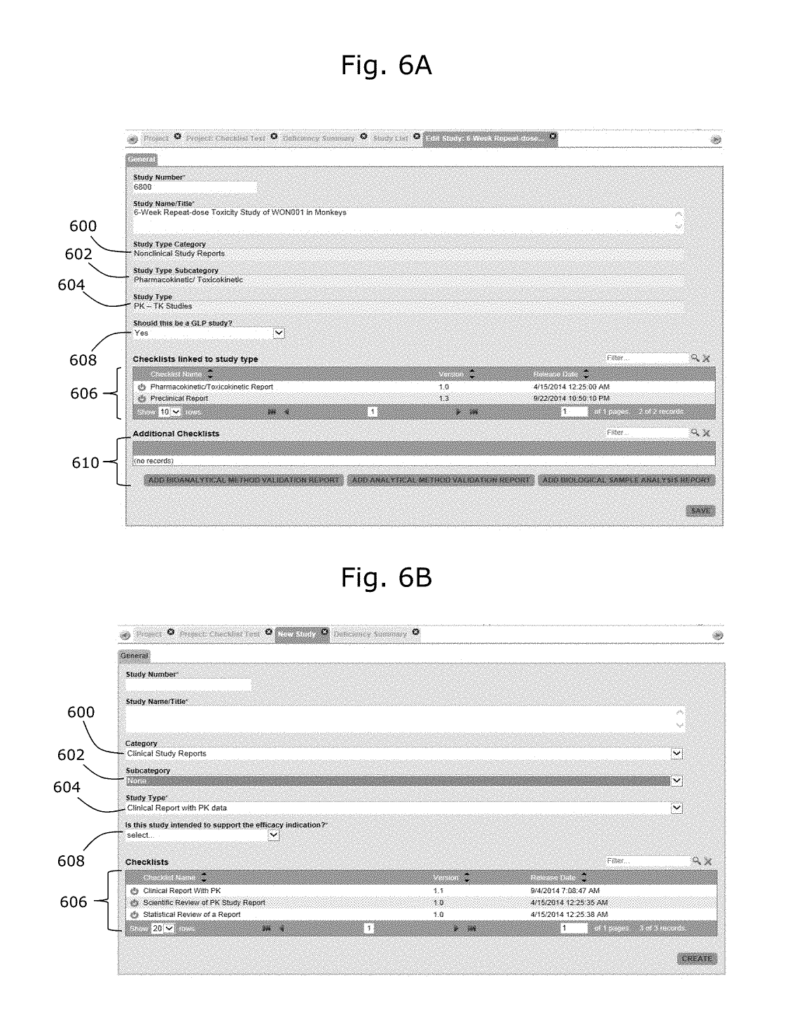

FIGS. 6A and 6B are examples of user interfaces that may be used in step 504 to manually add studies to a project. FIG. 6A shows an entry interface for a new nonclinical study, and FIG. 6B shows an entry interface for a new clinical study. In each case, the administrator may be prompted to enter a category 600 and subcategory 602 for the study to help identify and sort the studies. The administrator is also prompted to enter a study type 604 for the category, which is selected from a predetermined list of study types available from a dropdown menu. Upon selecting a study type, the system automatically populates the study with one or more checklists 606 associated with the study type. The selection of checklists associated with each particular study type preferably is performed by the system, and the administrator and other users generally cannot change these associations, other than by adding additional checklists on a case-by-case basis.

The user interface also includes a criticality question 608 that is presented based on the user's selection of the study type. If the administrator selects a nonclinical study type, the interface appears with the criticality question in FIG. 6A, which may appear as a prompt to answer whether the study should be designated as a Good Laboratory Practices (“GLP”) study. If the administrator selects a clinical study type, the interface appears with the criticality question 608 in FIG. 6B, which may appear as a prompt to answer whether the study is intended to support the efficacy indication of the drug. The criticality question 608 may be used to determine the level of criticality that should be assigned to any deficiency with the information provided for the particular study.

The criticality question 608 also may be used to prompt the administrator to add more checklists to the study, or to automatically add additional checklists to the study. In this example, answering “Yes” to either question assigns a high level of criticality to any deficiencies, and prompts the addition of additional checklists. The prompt to add additional checklists is shown in FIG. 6A, in which the administrator has answered “yes” to indicate that the study is intended to be a GLP study. In this example, when “yes” is selected from the dropdown menu, the interface is reconfigured to include one or more additional prompts 610 to add new checklists, such as a bioanalytical method validation report, an analytical method validation report, or a biological sample analysis report. Upon selecting and saving the entry, the system will add the selected checklist(s) to the study.

The use of the criticality question is expected to provide significant benefits to the system. For example, it provides a simple and intuitive way to categorize the importance of any deficiencies with the supporting information (as explained in more detail below), without relying on particular user's potentially subjective interpretation of any given deficiency. Instead, the administrator only needs to make the relatively objective decision as to whether the study is a GLP study or is intended to support the efficacy indication. Using the criticality question to add checklists also avoids the possibility that checklists with more detailed questions that are appropriate for, say, a GLP study, are not used to analyze a non-GLP study, which may result in a large number of potentially irrelevant deficiencies being identified for the study. Is will also be appreciated that some study types may not require a criticality question. For example, a study type that is not related either to GLP studies or to supporting the efficacy indication may not require a criticality question.

As noted above, the system preferably is also configured to allow an administrator to import a number of studies, as depicted in step 506 of FIG. 5, from a properly formatted source file, such as a spreadsheet in Excel format or the like. Upon importing the studies, the system also preferably prompts the administrator to answer the same criticality questions 608 that would have been presented if the studies were manually entered.

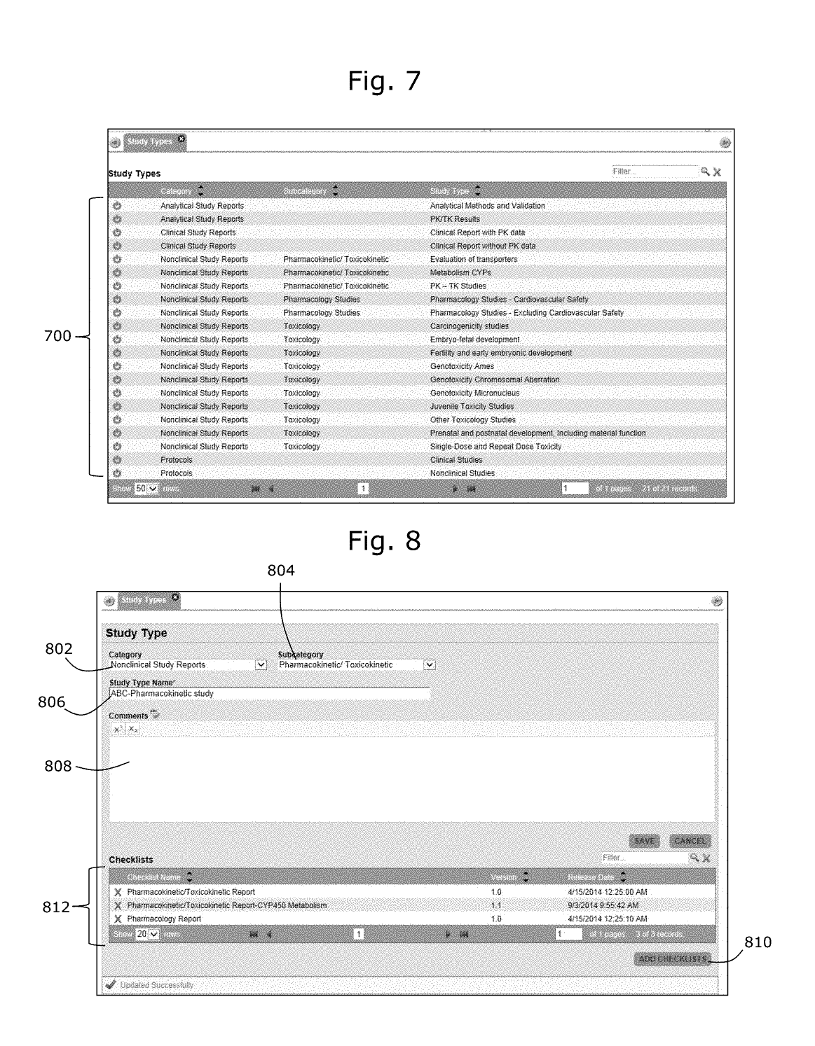

The system preferably includes a predetermined list of study types. Each study type preferably is unique, and has a unique associated set of checklists (checklists can, however, be common to multiple study types). FIG. 7 illustrates an exemplary list of twenty-one study types 700 that are provided in the system. These exemplary study types include, but are not limited to: an Analytical Methods and Validation study type, a PK/TK Results study type, a Clinical Report with PK data study type, a Clinical Report without PK data study type, an Evaluation of transporters study type, a Metabolism CYPs study type, a PK-TK studies study type, a Pharmacology Studies—Cardiovascular Safety study type, a Pharmacology Studies—Excluding Cardiovascular Safety study type, a Carcinogenicity studies study type, an Embryo-fetal development study type, a Fertility and early embryonic development study type, a Genotoxicity Ames study type, a Genotoxicity Chromosomal Aberration study type, a Genotoxicity Micronucleus study type, a Juvenile Toxicity Studies study type, an Other Toxicology Studies study type, a Prenatal and postnatal development, including material function study type, a Single-Dose and Repeat Dose Toxicity study type, a Clinical Studies study type, and a Nonclinical Studies study type. Other and alternative study types may be used in other embodiments. Each study type 700 is identified by a name (the third column), and also may be assigned a descriptive category (first column) and subcategory (second column) to aid with organization, searching and sorting. The category and/or subcategory identifier also may be used to indicate what criticality question may be associated with any particular study type, but it is not necessary for every study type to have an associated criticality question.

The list of study types, checklists, and associations between the study types and the checklists preferably cannot be changed by end users or administrators. Instead, the study types, checklists, and associations preferably are programmed into the system such that they can only be changed by the program developer (e.g., operating under “super user” credentials), or by formal upgrades to the system that are made by the program developer. FIG. 8 illustrates a user interface that may be used to create a new study type, and associate checklists with the new study type. Here, the user selects a category 802 and subcategory 804 for the study type using dropdown menus, and adds a unique study name 806. The user is also provided a free text field 808 to add comments to describe, for example, the reason for or purpose of the study type. In this and other free text fields, the system may implement a spell-checking system, as known in the art, which may allow the administrator to select the preferred language (e.g., British English, American English, French, etc.). The interface also includes a prompt 810 to add checklists, and a list of checklists 812 that already have been associated with the study type. The checklists 812 may be identified by name (first column), version (second column) and release date (third column), or by other suitable indicia.

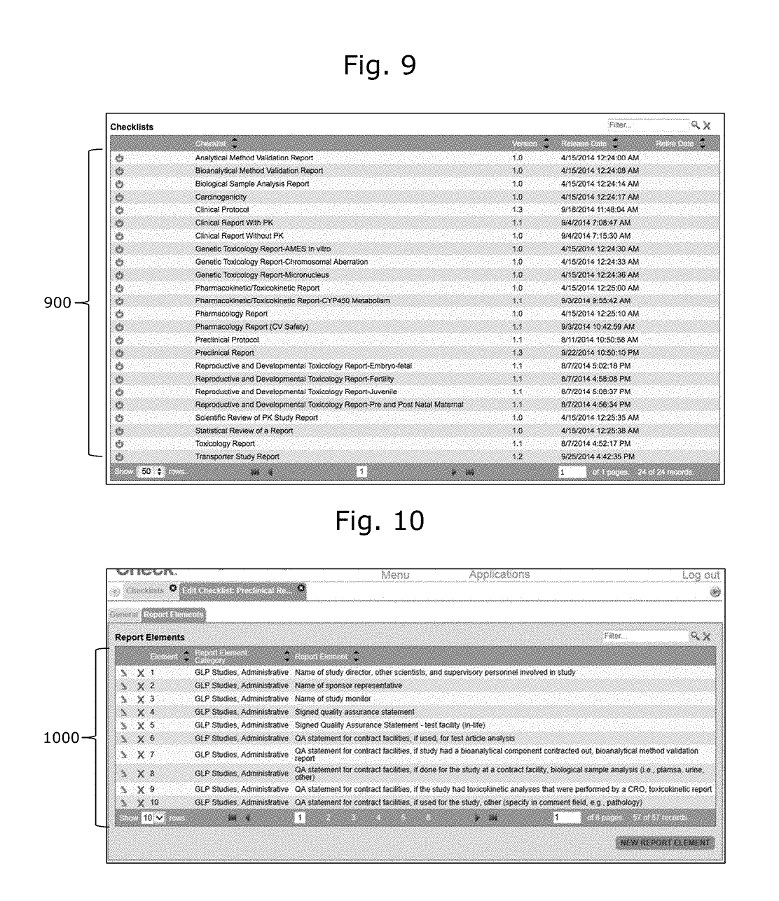

The system preferably includes a predetermined list of checklists. For example, the system may include twenty-four unique checklists 900, as shown in FIG. 9. Each checklist may have a unique name (first column), version (second column) and release date (third column). In the present example, the checklists include, but are not limited to: an Analytical Method Validation Report checklist, a Bioanalytical Method Validation Report checklist, a Biological Sample Analysis Report checklist, a Carcinogenicity checklist, a Clinical Protocol checklist, a Clinical Report With PK checklist, a Clinical Report Without PK checklist, a Genetic Toxicology Report-AMES in vitro checklist, a Genetic Toxicology Report-Chromosomal Aberration checklist, a Genetic Toxicology Report-Micronucleus checklist, a Pharmacokinetic/Toxicokinetic Report checklist, a Pharmacokinetic/Toxicokinetic Report-CYP450 Metabolism checklist, a Pharmacology Report checklist, a Pharmacology Report (CV Safety) checklist, a Preclinical Protocol checklist, a Preclinical Report checklist, a Reproductive and Developmental Toxicology Report-Embryo-fetal checklist, a Reproductive and Developmental Toxicology Report-Fertility checklist, a Reproductive and Developmental Toxicology Report-Juvenile checklist, a Reproductive and Developmental Toxicology Report—Pre and Post Natal Maternal checklist, a Scientific Review of PK Study Report checklist, a Statistical Review of a Report checklist, a Toxicology Report checklist, and a Transporter Study Report checklist. Other and alternative checklists may be used in other embodiments. As noted above, each study type is associated with one or more checklists, and any particular checklist can be associated with one or more different study types. The checklists also may be categorized into different groups. For example, each checklist might be categorized as either regulatory (e.g., referring to formal regulatory requirements) or technical (e.g., referring to scientific collection and analysis of data). Also as noted above, it is preferred that only the developer or super users can change, add or retire checklists

Each checklist includes any number of (i.e., one or more) elements, and it is expected that in many cases each checklist will include a large number (dozens or hundreds) of elements. Some elements may be present in multiple different checklists. Each element comprises a feature that the regular user and final reviewer should identify and evaluate in the supporting study information, to determine whether the feature is present and compliant with any existing requirements. Examples of some elements are shown in FIG. 10, which shows an exemplary interface that may be used to edit the elements of a checklist. This interface presents a list of elements 1000 associated with one particular checklist. Each element may have a title (third column) to uniquely identify the element, and a category (second column) that may be used to help organize, sort and search elements.

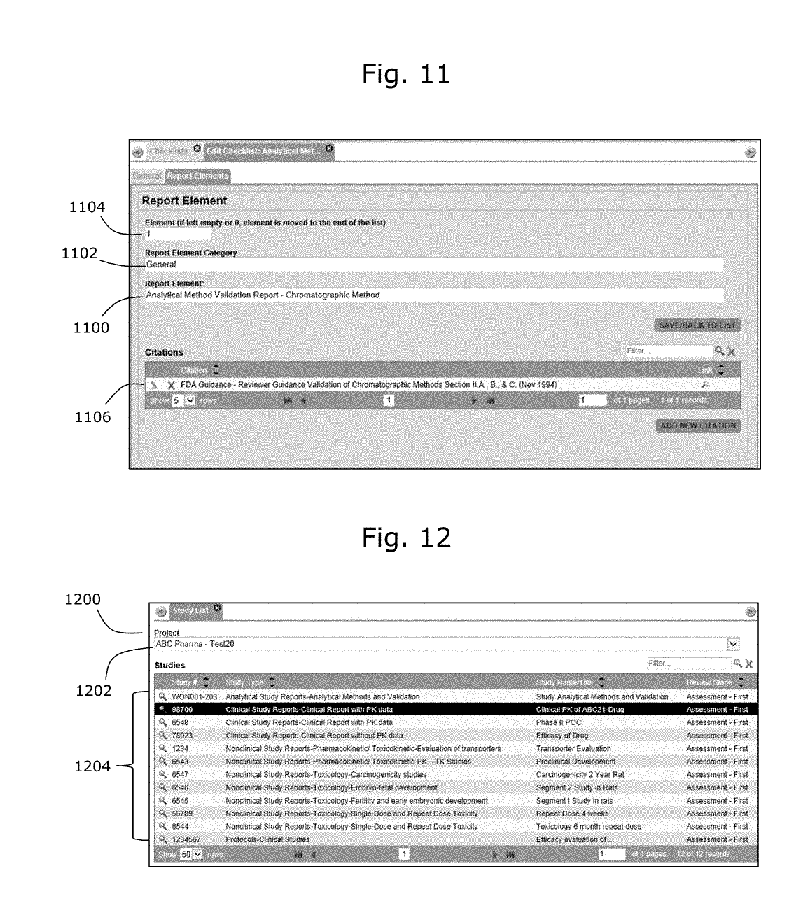

FIG. 11 shows an example of an interface for viewing or editing the details of an element. (As with the study types and checklists, it is preferred that only super users or developers are able to add, change or retire elements.) Here, the report element is identified by a unique name 1100, and includes a category designation 1102, and an element number 1104 to specify the element's location in the checklist. The name 1100 preferably is sufficiently descriptive to explain to the regular user or final reviewer what it is that he or she is supposed to look for in the supporting data. However, a further textual description of the feature that is intended to be reviewed also may be provided.

Each element also preferably has one or more associated citations 1106 to identify regulatory or legal basis or guidelines for the element. For example, in the shown interface, the element includes a citation to the FDA's issued guidance on how to review validation of chromatographic methods. The citation 1106 preferably also includes a hyperlink 1108 that the user can select to direct the user to the citation source. Such a hyperlink 1108 may direct the user's computer to a static link (i.e., a file stored in the system or on an external server 117₁-117ₙ that is called up when the hyperlink 1108 is selected), to a dynamic link (i.e., a link to another entity's stored data, such as a link to the FDA's webpage hosting the latest versions of 21 C.F.R.), or to any other source of the citation. In a preferred embodiment, every element includes a citation 1106, but this is not strictly required.

The system may be configured to allow special releases of new study types, study type checklist associations, checklists, elements, and citations to guidance. Such changes may be desirable to account for changes in the controlling law or guidance, to correct errors, or to add additional functionality (e.g., new releases for different regulatory bodies). Special releases may be provided in the form of pushed updates (updates that are automatically sent to the system), manually downloaded updates, and so on. The release may be made immediately upon its preparation, or saved for a periodic (e.g., annual) update. Such upgrades may be provided with release notes to explain the new study type. Upon adding a new study type, checklist, element, etc., it also may be necessary to retire a previous version of the same to prevent further use of the outdated version. In order to account for ongoing user activity, the special release may be conducted by allowing users to check in the old version of a checklist that has been updated, but then retiring that version of the checklist and making it unavailable for further access. The system also may be configured to poll all projects in progress, and provide a summary of any previously-completed checklists that are out of date following the special release. The system also may be configured to require previously-entered information to be reentered to ensure that the analysis of the elements is up to date. Alternatively, the previously-entered information may be retained without reentering, and then reviewed to ensure that any changes made by the special release do not affect the outcome of the analysis. The acts of creating new study types, checklists, elements, etc. and retiring old version of the same preferably are performed only by a central programming authority or super user, to make sure that all versions of the system are operating with the same parameters.

Referring back to FIG. 5, after the administrator sets up a new project, he or she may release the project, as shown in step 508, to allow regular users and final reviewers to start reviewing the study checklists. The administrator may release the entire project when all of the studies and checklists are completely entered, or he or she may serially release the studies and checklists individually or in groups as they are added to the project. The release function may be provided, for example, in the dropdown menu 404 within the Administrative Functions application (see FIG. 4). The initial release begins the first review stage, in which reviewers (e.g., regular users and final reviewers) are able to check out the checklists for review.

The reviewers may access the system using the same login portal as the administrators, but their access credentials may prevent access to certain applications. For example, a reviewer may not have access to the reports module 304, and the main dropdown menu 404 may include access only to the Study List and Program Level Guidance applications.

The Study List application directs the reviewer to a list of projects, studies or checklists to which the particular reviewer has access. For example, in some cases, a reviewer may have access to all of the projects and all of the study types associated with one sponsor, but may not have access to another sponsor's projects. As another example, a reviewer may have access to checklists that are categorized as regulatory, but not to checklists that are categorized as technical. Other distinctions may be implemented in other embodiments, as will be appreciated by persons of ordinary skill in the art.

FIG. 12 shows an exemplary interface that is presented to the reviewer upon activating the Study List application. The interface 1200 includes a dropdown menu 1202 to select a project, and a list of studies 1204 associated with the selected project. Each study 1204 may be identified by a study number (first column), a study type (second column), a study name (third column), and a review stage (fourth column). The reviewer selects one of the studies, such as by clicking on the magnifying-glass icon, to select a study for review. Studies that are temporarily unavailable for review (e.g., those checked out by other reviewers) may be shown in the list, but preferably are not accessible to the reviewer for substantive review (reviewing in read-only mode may still be an option in this case).

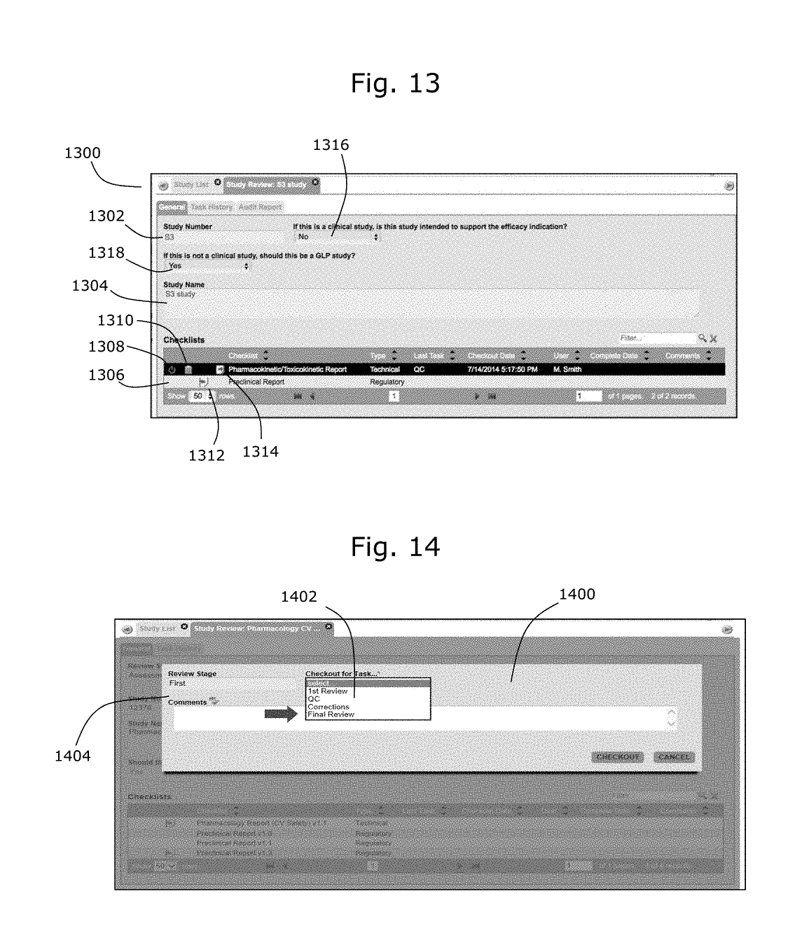

Upon selecting a study for review, the user may be presented with the exemplary interface shown in FIG. 13. This interface 1300 identifies the study number 1302 and study name 1304, and provides a list of checklists 1306 associated with the study. Each checklist 1306 may be identified by name (first column), and include one or more status indicators, such as the type of checklist (second column), the last task performed on the checklist (third column), and the last checkout date (fourth column), last user, completion date, comments, and so on. Each checklist 1306 also may include iconographic information or selection fields, such as an icon to open the checklist in read-only mode 1308, an icon to open the checklist in edit mode 1310, an icon to check out the checklist to the current user 1312, and an icon to check in the checklist to make it available to other users 1314. The interface 1300 also may include one or more criticality questions, such as a question whether the study is a clinical study intended to support the efficacy indication 1316 and a question whether the study is a non-clinical GLP study 1318. The values for these criticality questions may be pre-selected according to the answers that were provided by the administrator during the setup process, but the reviewer may be given an opportunity to change the answer after reviewing the supporting information in more detail. The reviewer also may be provided with other interfaces, such as an interface to review that particular user's task history, and the like.

After opening the study review interface 1300, the reviewer may select and check out a particular checklist for review. Upon doing so, the user reviewer may be receive a prompt 1400 to select the task that the reviewer will be performing on the checklist. An example of such a prompt is shown in FIG. 14. Here, the system requires the reviewer to select from a menu 1402 whether the task is for 1^{st} Review, QC (quality control), Corrections, or Final Review. The types of task may be limited according to the user's particular credentials. For example, a regular user may not have access to the Final Review task. The prompt 1400 also may indicate the project's review stage 1404, as explained in more detail below.

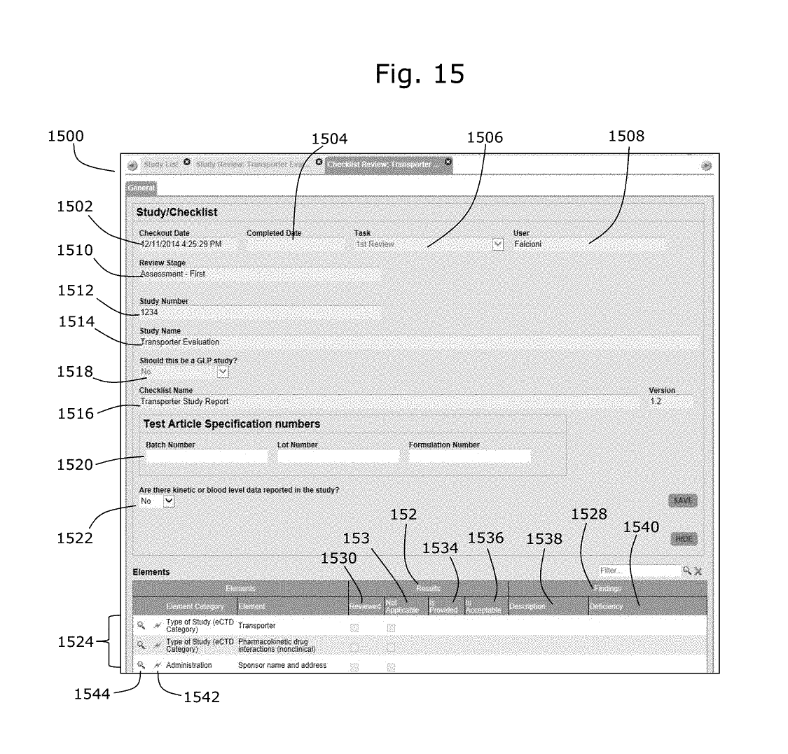

Referring to FIG. 15, upon selecting the task, and checking out the checklist, the reviewer may be presented with a checklist overview interface 1500. This interface 1500 includes bibliographic details, such as the checkout date 1502, completed date 1504, task 1506, user 1508, review stage 1510, study number 1512, study name 1514, and checklist name 1516. The interface also may include a criticality question 1518, which provides another opportunity for the reviewer to identify whether the study is a clinical study intended to support the efficacy indication, or a non-clinical GLP study. The checklist overview interface 1500 also may include entries for the test article specification numbers 1520 (e.g., batch number, lot number and formulation number).

The interface 1500 preferably also includes a study level query 1522 asking whether kinetic or blood level data is reported in the supporting information. The purpose of the study level query 1522 is to identify when a report on such data exists in the documentation, which can affect the importance or relevance of the study or how the information is interpreted by a regulatory authority. As noted above, in some cases the administrator may not be aware of the full contents of the supporting information, as it can include many hundreds or thousands of pages of documents. Providing the reviewer the opportunity to identify whether there is kinetic or blood level data in the supporting information allows the system to reconfigure to properly evaluate the supporting information. The reviewer preferably can change the answer to the study level query 1522 at any time during review of the checklist.

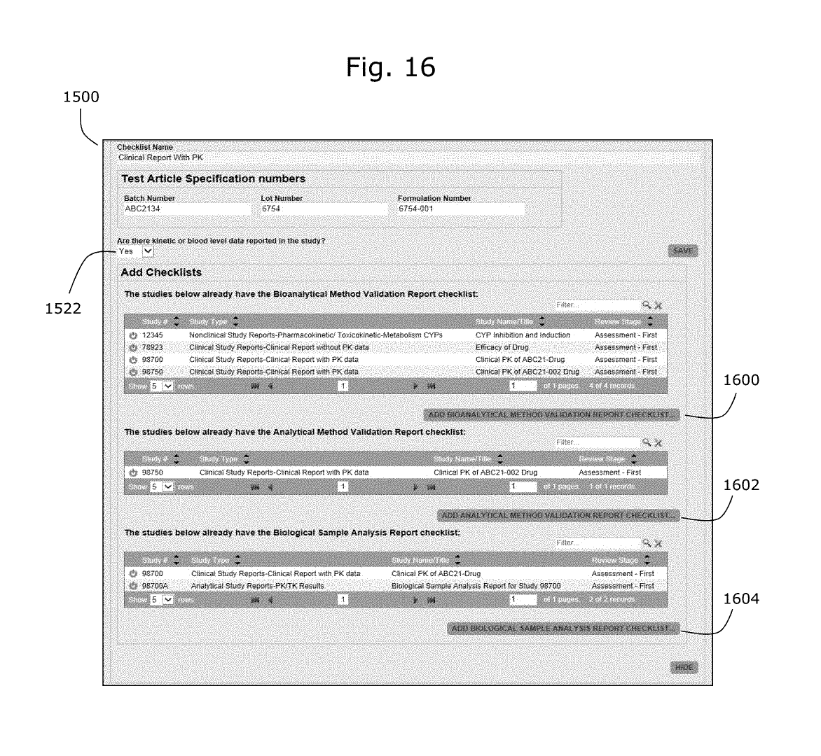

When a reviewer changes the study level query 1522 to “yes”, the system preferably automatically adds additional checklists to the study, or prompts the user to add checklists to the study. For example, as shown in FIG. 16, the checklist overview interface 1600 may be reconfigured have the user acknowledge that the new checklists have been added (in systems where the checklists are added automatically), or to prompt the reviewer to add one or more checklists to the study. The additional checklists also may be added without prompting or notifying the user. These additional checklists may include, for example, a bioanalytical method validation report checklist 1600, an analytical method validation report checklist 1602, or a biological sample analysis report checklist 1604. The prompt may also indicate whether the suggested additional checklists have already been added to the study or project. For example, in FIG. 16, the three suggested additional checklists have already been added to other studies for the same project. Presenting the reviewer with the list of other inclusions of the suggested checklists can give the reviewer the opportunity to evaluate whether it is necessary to add the suggested checklists to the present study.

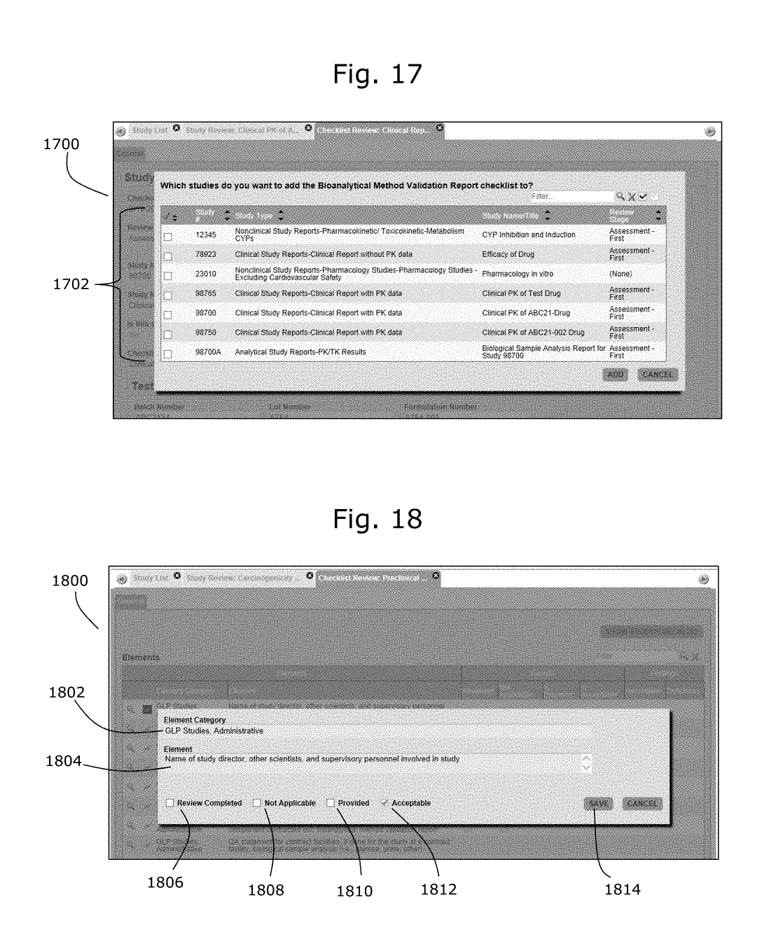

Upon answering the study level query 1522 to identify that the supporting information includes kinetic or blood level data, the system also may be configured to prompt the reviewer to add the suggested checklists to other studies. For example, a prompt 1700 such as shown in FIG. 17 may be provided with a list of other studies 1702 that might require the suggested checklists. Each of the studies 1702 may be identified by study number, study type, study name, review stage, etc. Providing this prompt is expected to allow the reviewer to quickly and easily account for the discovery of the kinetic or blood level data, and the effect that this data can have on the various studies that are part of the project.

Referring back to FIG. 15, the checklist overview interface 1500 also may include a list of individual elements 1524 associated with the checklist. Each element 1524 may be identified by an element category (first column) and an element name (second column). Each element also may include one or more associated variables, which may be organized as results 1526, or findings 1528. For instance, in the results field 1526, each element 1524 may include a review complete variable 1530 to indicate whether the reviewer has completed review of the element, a not applicable variable 1532 to indicate that the element is not applicable to the supporting information, a provided variable 1534 to indicate that the information identified in the element is provided in the supporting information, and an acceptable variable 1536 to indicate that the information provided in the supporting information is acceptable. The findings field 1528 may include a description field 1538 and a deficiency field 1540, which may show a user-provided description of any deficiencies, and a user-selected predetermined deficiency description, as explained in more detail below.

The system may be configured in any suitable way to receive the reviewer's evaluation of the supporting information. In the shown example, a speed entry icon 1542 is provided to allow the reviewer to quickly identify an element as either not applicable or acceptable, and an extended entry icon 1544 is provided to allow the reviewer to perform a more detailed data entry process for other elements.

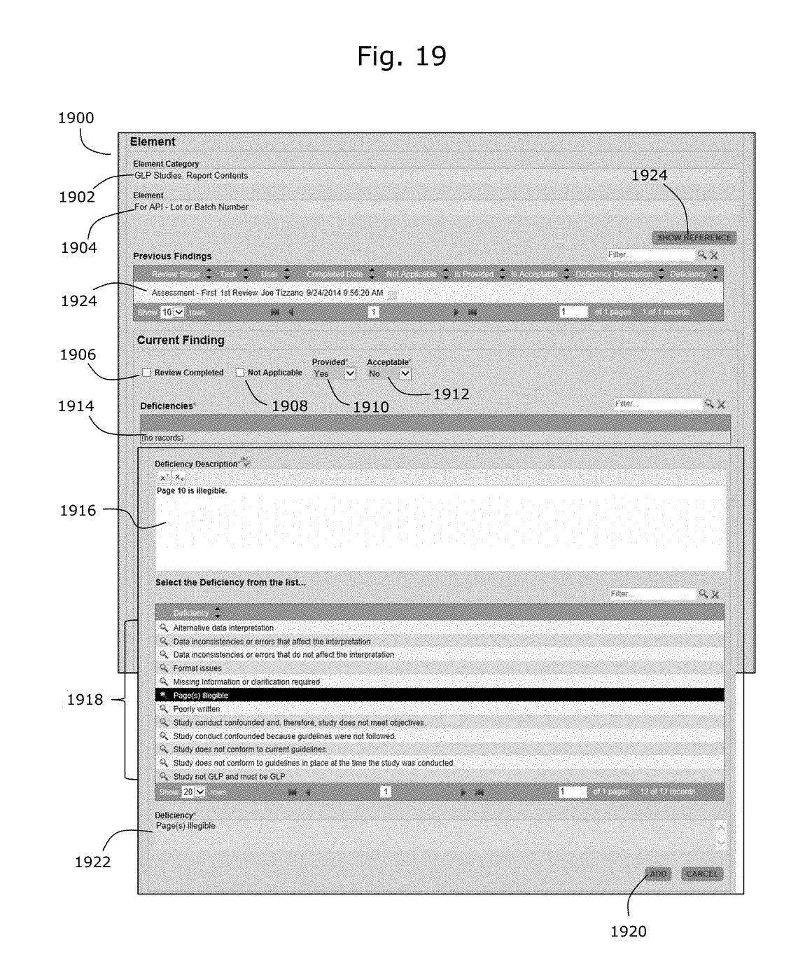

FIG. 18 shows an example of a speed entry prompt 1800 that is displayed when the reviewer selects the speed entry icon 1542 for an element. The prompt 1800 identifies the element category 1802 and element name 1804, and includes four selection boxes: a review completed query box 1806, a not applicable query box 1808, a provided query box 1810, and an acceptable query box 1812. These correspond to the four variables 1530, 1532, 1534, 1536 identified above. The prompt 1800 is presented in a default configuration in which the acceptable query box 1812 is checked, and the remaining boxes are not selected. The reviewer can alternatively select the not applicable query box 1808, in which case the acceptable query box 1812 may remain checked or automatically be unchecked. Upon selecting the save icon 1814 the review completed query box 1806 may be automatically checked, the reviewer's selection is recorded into the system, and the element will thereafter appear with the appropriate variable 1530, 1532, 1534, 1536 marked according to the reviewer's selection. Such an element may be categorized by the system as being satisfied. Alternatively, the reviewer may be required to manually check the review completed query box 1806 before being allowed to select the save icon 1814. It will be appreciated that, upon selecting either the not applicable query box 1808 or the acceptable query box 1812 and selecting the save icon 1814, the user has effectively replied to all four queries, because either of those replies renders the remaining queries moot. However, it may be desirable to require the user to manually select the review completed query box 1806 to maintain a complete audit trail of the user's work.

FIG. 19 shows an example of an extended entry prompt 1900 that is displayed when the reviewer selects the extended entry icon 1544 for an element. This prompt 1900 identifies the element category 1902 and element name 1904, and includes a current finding entry area having a review completed query box 1906, a not applicable query box 1908, a provided query dropdown menu 1910 with “yes” and “no” as possible answers, and an acceptable query dropdown menu 1912 with “yes” and “no” as possible answers. The prompt 1900 also may present a list of deficiencies 1914 that are associated with the element. As with the speed entry prompt 1800, the user may finalize the element review by saving the replies to the query boxes and other queries associated with the element, and doing so effectively acts as a reply to all of the queries in those cases where one reply (e.g., “not applicable”) may moot the need to enter replies to the other queries, or it is otherwise unnecessary to manually reply to each query in the element.

When a reviewer selects “no” in the acceptable dropdown menu 1912, the prompt 1900 allows the reviewer to add one or more deficiencies. For example, the prompt 1900 may provide a deficiency description query field 1916 into which the reviewer can manually type the nature of a deficiency (e.g., “Page 10 is illegible”). For each deficiency, the reviewer preferably also must respond to a query to select a deficiency type (e.g., “Page(s) illegible”) from a predetermined list of deficiency types 1918. After typing a description and selecting a deficiency type, the reviewer may select an add icon 1920 to add the deficiency to the element. If the element has multiple deficiencies, the reviewer can continue entering descriptions and adding deficiency types from the list 1918 until the review of the element is complete. The prompt 1900 may include a summary list 1922 of all of the deficiencies types added from the list 1918. The system preferably is configured to prevent the reviewer from leaving the extended entry prompt 1900 if “no” is selected in the acceptable dropdown menu 1912, until the reviewer changes the selection to “yes” or adds one or more deficiencies.

The use of a predetermined list of deficiency types is expected to provide a significant benefit to some embodiments. For example, requiring reviewers to select deficiency types from a list can help harmonize the manner in which elements are reviewed by different reviewers, and can help each reviewer better understand what constitutes a deficiency for a particular element. The use of a predetermined list of deficiency types also allows the system to easily identify remedial steps that may be taken to address each deficiency type, and generate a report listing all of the deficiencies and suggested remedial actions. This aspect of the system also helps facilitate various useful data analysis processes. For example, if a sponsor provide supporting data that includes a large number of a particular deficiency type, it may indicate a systemic problem with the sponsor's drug development program. As another example, the performance of individual reviewers can be assessed by examining their practices for assigning deficiencies to the elements. Other uses will be readily apparent to persons of ordinary skill in the art in view of the present disclosure.

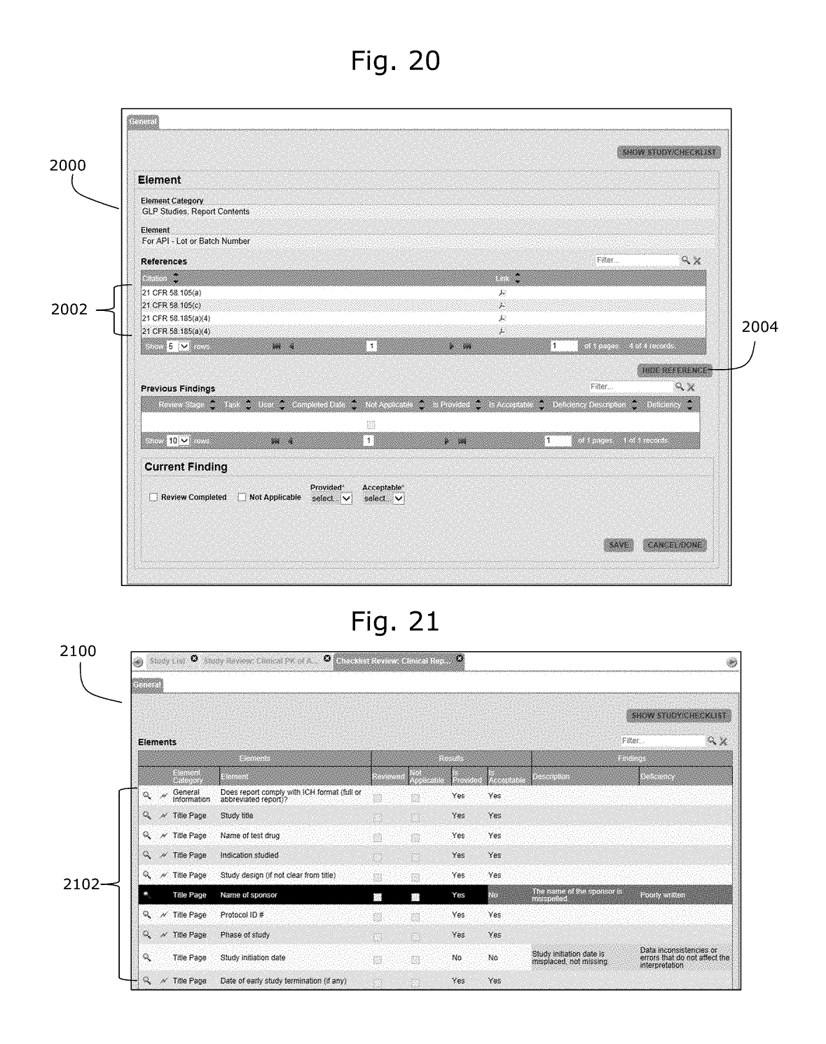

The extended entry prompt 1900 also may include other features, such as a list of previous findings 1924 that have been made in relation to the particular element. The extended entry prompt 1900 (and the speed entry prompt 1800) also may include a show reference icon 1926 that may be selected to provide guidance to the reviewer regarding the purpose and proper analysis of the element. FIG. 20 illustrates how the extended entry prompt 1900 may be reconfigured when the reviewer selects the show reference icon 1926. (It will be appreciated that, instead of reconfiguring the prompt 1900 itself, a separate window or other user interface may be opened.) The reconfigured prompt 2000 provides a list of one or more citations 2002 to guidance regarding the element. Each citation 2002 may include a citation name (first column) and a hyperlink (second column) to the relevant regulatory or legal basis or guidelines for the element. Upon selecting the hyperlink, the system may open a new window or other interface to direct the reviewer to the relevant law, code (e.g., the relevant section of 21 C.F.R.), FDA guidance document, or the like. The hyperlink preferably is also configured to go directly to the relevant part of the relevant citation. Furthermore, where the system has control of the contents of the citation (e.g., it is stored on an internal server), the citation can be annotated or highlighted to make the relevant citation even more clear to the reviewer. For example, selecting the hyperlink may open a portable document file (“pdf”) document that includes a section of 21 C.F.R., with the relevant part of the section highlighted. The reconfigured prompt 2000 also may provide an embedded written description (e.g., a text box) providing the relevant guidance. The guidance associated with each element may be set up as described previously herein, or otherwise provided in the system. When the reviewer no longer wishes to view the guidance, the reviewer may select a hide reference icon 2004 to return to the original extended entry prompt 1900.

The inclusion of direct access to relevant guidance for each element is a significant improvement over earlier simple spreadsheet-type lists of elements that have been used in the past to help evaluate information intended for regulatory review. By associating hyperlinks to the relevant guidance with the element review prompt, the reviewer is no longer required to manually identify relevant guidance to assess the various elements of a study. This greatly reduces the possibility that the reviewer will review and rely on outdated or incorrect guidance, and can save a significant amount of time. Providing the guidance in this manner also allows the reviewer to self-educate on the important details of the relevant law or guidance.

As noted above, the study type defines the checklists that are to be used during the review stage. However, not all information about a study may be known at the time the study is created by the administrator. This is not an uncommon problem, because a study's supporting information often includes many thousands of pages of documentation, and as a practical matter the administrator cannot always accurately prepare a checklist for use by the reviewers until the contents of the study have been fully reviewed. One possible solution to this problem would be to simply present every possible checklist (including every possible element that might be required by the regulatory authority) to the reviewer. This “brute force” method would be likely to ensure that all regulatory requirements are considered, but this process also would be very time-consuming, and the reviewers would be likely to have trouble keeping track of the large number of checklists and elements to make sure each is addressed.

To address this problem, the system may be configured to prompt the user to add additional checklists based on the answers to certain key questions. This foregoes the necessity to address certain checklists unless the supporting information invokes their consideration, which can save time and money, and prevent the appearance that the supporting information is somehow deficient for lacking the information required in the additional (but unnecessary) checklists.

Processes for adding new checklists have been described above in relation to adding checklists to a study based on the reviewer's answers to criticality questions and study level queries. Embodiments also may be configured to manually or automatically add checklists to a study (or elements to a checklist) based on the reviewer's replies to particular checklist elements. For example, certain checklists may include trigger elements that ask whether the supporting information includes particular data, and if the reviewer answers “yes” to any of these trigger elements, then the system can automatically add one or more additional checklists specific to these types of data to the study. As a more specific example, the “Pharmacokinetic/Toxicokinetic Report” and “Scientific Review of PK Study Report” checklists shown in FIG. 9 may include one or more elements that ask whether the supporting information includes metabolism and transporter drug interaction-related data, and if the answer is “yes” then the system automatically adds the appropriate additional checklist(s) (e.g., the Pharmacokinetic/Toxicokinetic Report—CYP450 Metabolism checklist and the Transporter Study Report checklist) to the study. As another specific example, the “Toxicology Report,” “Reproductive and Developmental Toxicology Report-Fertility,” “Reproductive and Developmental Toxicology Report-Embryo-Fetal” and “Carcinogenicity” checklists may include one or more elements that ask whether the supporting data includes toxicology-related data (e.g., there may be a question asking whether the toxicology repeat-does report contains safety pharmacology data), and if the reviewer answers “yes,” the system automatically adds the appropriate additional checklists(s) (e.g., the “Pharmacology Report (CV Safety)” checklist) to the study. In other embodiments, other trigger elements and associations between trigger elements and additional checklists may be used, and the system also may be configured to prompt the reviewer to manually add the necessary checklists, as described above in relation to the criticality questions and study level queries. Upon adding checklists in response to answers to trigger elements, the system or reviewer may also change a previously entered answer to the criticality question or study level query.

Any given study may include a number of checklists, and various checklists may include duplicative elements. As such, the system may be configured to allow the reviewer to skip elements that are duplicative to those that were previously answered in other checklists. For example, the system may be configured to show those duplicative elements in read-only mode, or it may be configured to show where each element has been previously answered so the reviewer can manually determine whether it is necessary to re-answer an element for a different checklist. Other alternatives will be readily apparent to persons of ordinary skill in the art in view of the present disclosure.

The reviewer completes the 1^{st} Review task by addressing all of the necessary elements in a checklist. After the checklist is complete, the reviewer checks the checklist back into the system, and the replies to the elements are recorded and stored. The 1^{st} Review results preferably are permanently stored in the database to allow auditing of the answers at a later date, and the checklist may be locked to prevent further reviews using the 1^{st} Review option shown in the prompt 1400 of FIG. 14. Checking in the checklist also allows the next task (e.g., quality control) to begin. Also upon checking in the checklist, the system may be configured to present a list of deficiencies associated with the checklist. Additional processing associated with the deficiencies is described subsequently herein.

As shown in FIG. 14, the next task in the exemplary embodiment is a Quality Control (“QC”) review. After a checklist is checked in, it is released to a second reviewer, who performs essentially the same process as the first reviewer to address and assess each element of the checklist. The second reviewer identifies satisfied or not applicable elements, and finds and describes any deficiencies that might be found. Upon completion, the second reviewer checks the checklist back in, where the findings are stored. The results of the Quality Control review may be permanently stored, or they may be editable to account for corrections that might be made during a subsequent Corrections task.

The system preferably also includes a Corrections task that may be activated after the Quality Control review. The Corrections task is used to identify differences between the 1^{st} Review and the Quality Control review. For example, the system may present an interface 2100 as shown in FIG. 21, to list each element 2102 and highlight those elements 2102 for which the 1^{st} Review and Quality Control review provided different replies. In this example, the interface is highlighting the “Is Acceptable,” “Description” and “Deficiency” fields for the “Name of sponsor” element to indicate that the two reviewers entered different answers for these aspects of this element. (Note that the remaining parts of the Name of sponsor element are highlighted black, but this is simply because this element is currently being selected.) Similarly, the “Description” and “Deficiency” fields of the “Study initiation date” element are highlighted to indicate a difference between the reviewer's answers to these parts of this element. The system can readily identify differences by matching data entries for each field, and performing text comparisons for free-text fields. The system may be configured to identify any difference, regardless of how trivial it might be (e.g., the second reviewer adds an “s” to a word in the “Description” field, but the entry is otherwise identical to the first reviewer's entry). Although not strictly required, this level of scrutiny is expected to provide a high level of review scrutiny to assure optimal results.

The Corrections task may be used for various purposes. For example, this may provide an opportunity for reviewers to consult and determine whether there is a true discrepancy between their reviews, or whether one reviewer or the other made a mistake. After consulting about any discrepancies between the two reviews, if the reviewers agree to a corrective action, corrections may be made to the relevant element(s) to bring the two reviews into agreement. Corrections may take the form of an annotated record that is associated with the particular element in question, by revising the Quality Control record to make the change, or by other means, as will be appreciated by persons of ordinary skill in the art.

Comparative data from the Corrections task also may be used to evaluate reviewer performance, to assist with training, and so on.

The system may be configured to allow various administrative functions throughout the review process. For example, a record may be kept of each recorded entry for each element (including any correction) to assure a complete audit trail for the study. Such records may include a user identity, edit time, substance of the edit, and so on. The records also may include keystroke-level records to track each individual interaction with the system. The system also may be configured to allow oversight functions, such as the ability to examine checklists in read-only mode even while they are checked out, to actively monitor the review process.

As noted above in relation to FIG. 4, the system may include access to a Program Level Guidance application. Opening this application provides the user with access to guidance documents intended, for example, to assist with understanding and performing the review process. For example, the Program Level Guidance documents may comprise general regulatory information that is potentially relevant to the study as a whole. Such information may markedly modify, or emphasize in a different way, the evaluation of elements in one or more study checklists. For example, the ICH S6 and addendum guidance documents from the FDA are specific to the development of biotechnology-derived products, and these guidance documents substantially modify the ICH M3(R2) guidance recommendations concerning toxicity investigations related to genotoxicity, carcinogenicity, and reproductive and developmental toxicity. As such, the ICH S6 and addendum guidance documents may be provided for review in particular studies. The Program Level Guidance may be supplemental to the guidance that is provided for each particular element as explained above, but the Program Level Guidance may include information that overlaps with the guidance provided for each element.

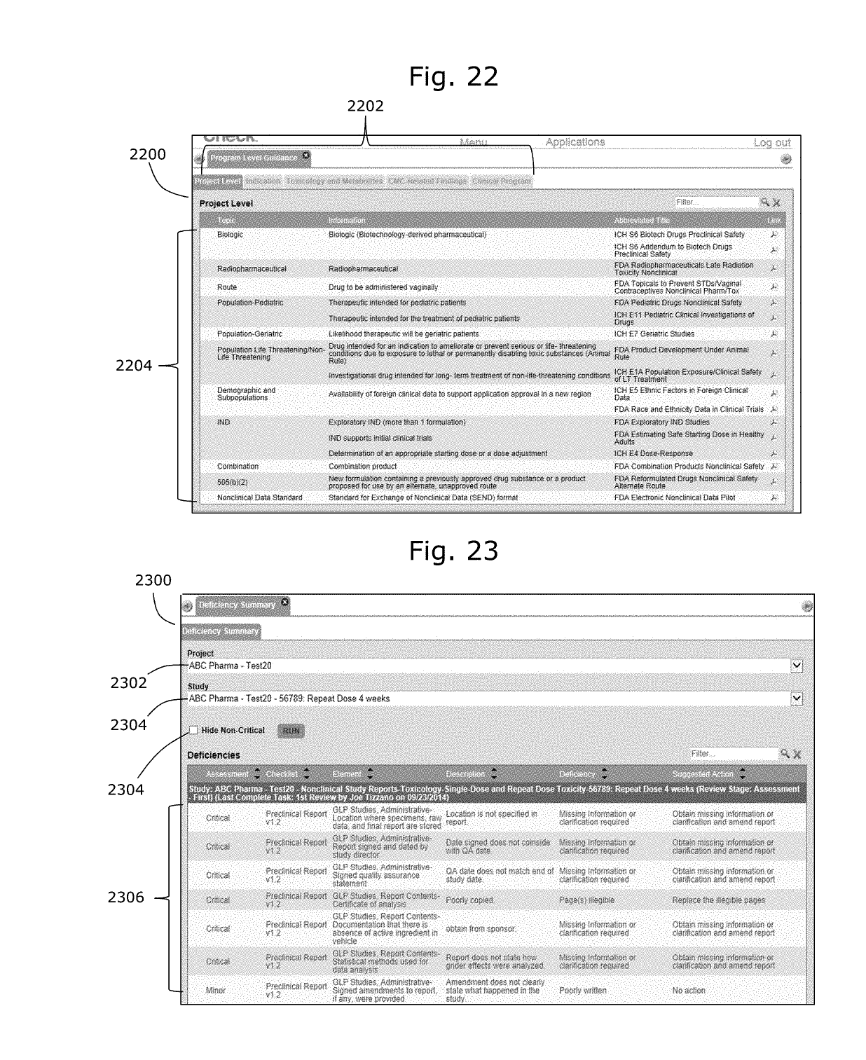

FIG. 22 shows an example of a Program Level Guidance interface 2200. The interface 2200 includes five tabs 2202, labeled “Project Level,”“Indication,”“Toxicology and Metabolites,” “CMC-Related Findings,” and “Clinical Program.” Each tab 2202 provides a list of relevant guidance documents 2204, and each document 2204 may include an associated topic (first column), informational description (second column), abbreviated title (third column), and hyperlink to the relevant document (fourth column). An administrator or developer may associate the particular desired Program Level Guidance documents with each checklist, study, or sponsor. Preferably all users can access the program level guidance.

After the 1^{st} Review, Quality Control review and Corrections tasks are complete, the checklists are released for Final Review. The Final Review may be used to evaluate the previous review activities, identify any open questions related to the review process, and obtain an overall evaluation of the review status. The Final Review task preferably can only be performed by users with final reviewer-level credentials, administrators, or other higher-level users.

In one embodiment, Final Review may be initiated by using the Study List application in the user interface 400 (see FIG. 4) to open one or more projects, studies, or checklists under the Final Review task (see FIG. 14). The projects, studies or checklists may be opened in a read-only mode to prevent inadvertent modification of the contents. Once the projects, studies or checklists are open, the Final Reviewer can start the Deficiency Summary application through the user interface 400. The Deficiency Summary provides a detailed summary of all of the deficiencies of the selected projects, studies or checklists. The system may be configured such that the Deficiency Summary is only be accessible for checklists that have been checked in. If necessary, the Final Reviewer or Administrator may be provided with the ability to force the check in of checked out checklists.

FIG. 23 provides one example of a Deficiency Summary report 2300. This report is a Deficiency Summary for a nonclinical GLP study report (in particular, a Single-Dose and Repeat-Dose Toxicity report) that was evaluated using the Preclinical Report checklist. The report 2300 identifies the project name 2302 and study name 2304, and provides a list of deficiencies 2306 that were found for particular elements during the review process. Each deficiency 2306 is identified by a criticality assessment (first column), a checklist in which the deficiency was found (second column), a particular deficient element (third column), a reviewer's description of the deficiency (fourth column), a predetermined deficiency type (fifth column), and a suggested action to address the deficiency (sixth column). The report 2300 preferably is sortable and searchable, and may include an icon 2308 to hide non-critical or less important deficiencies.

The criticality assessment indicates the importance of the deficiency in relation to obtaining approval of the subject of the study (e.g., the drug or device). In this example, the assessments are identified as “critical” and “minor.” Different colors may be used to identify each type of deficiency (e.g., red for “critical,” green for “minor,” and blue for “major”). As explained above, the criticality question 608 may be used to determine the level of criticality that should be assigned to any deficiency for a given study. In the example of FIG. 23, this study was identified (during initial setup or review) as being intended to be a GLP study. GLP studies are required to follow the GLP regulations set forth in 21 C.F.R. part 58, and a failure to follow certain of these rules is likely to bar approval of the drug that is the subject of the study. Thus, the system is configured to identify certain deficiency types for certain elements for GLP studies as being “critical” (deficiencies that affect the acceptability of the document or program to health authorities). If the deficiency type is not a type that could affect acceptance, such as errors in the text, tables or figures or errors in editing or formatting, the system designates it as “minor.” In this case, six elements have deficiencies that are critical enough to affect the approval of the drug, and one deficiency is a minor deficiency that would not affect the approval of the drug.

If this were not a GLP study, the system could be configured to designate the six “critical” elements as being “major,” but the “minor” element would still be designated as “minor,” because the “minor” element has little or no bearing on acceptance of the study regardless of whether it is a GLP study. This type of system can be readily established by associating a risk value with each type of deficiency, and categorizing some deficiency types as being high risk (which would be identified either as “critical” or “major” when present), and others as being low risk (which would always be identified as “minor”). Of course, other embodiments could use additional levels of risk assessment, and other embodiments also could use more complex categorization schemes, such as by categorizing some deficiencies as being “minor” in the context of a non-GLP study but critical in the context of a GLP study. Other alternatives will be readily apparent to persons of ordinary skill in the art in view of the present disclosure.

From the foregoing, it will be appreciated that the decision to designate a deficiency as critical can be based on several different variables. In one embodiment, there are two variables: first, the study type must be designated as a GLP study (this can happen during initial setup or during review, as explained above); and second, the reviewer must select a particular deficiency type from a predetermined list of deficiency types that is sufficient to affect regulatory approval. Using this logic, if the study that is the subject of the example of FIG. 23 were not intended to be a GLP study, then the system could instead designate the “critical” deficiencies as being either “major” (errors and problems that are significant but are not sufficiently serious to affect the acceptability of the document or program), or “minor,” because the first variable (GLP study) would not be satisfied to elevate the criticality assessment to the “critical” level.

In other embodiments other variables may be used or added. For example, a third variable may be that the deficiency must be with one or more particular elements in the checklists (e.g., a particular deficiency type, such as “page(s) illegible” may be a critical deficiency for some elements of the checklist, but not for other elements). Other alternatives will be readily apparent to persons of ordinary skill in the art in view of the present disclosure. The system is configured to identify the criticality assessment based on a review of the selected variables, which may be readily accomplished using database associations and logic.

As similar regime preferably is used for clinical studies that are intended to support the efficacy indication of the drug. If a clinical study is intended to support the efficacy indication, then certain deficiency types will result in a “critical” criticality assessment in the report. Alternatively, if a clinical study is not intended to support the efficacy indication, then those same deficiency types will result in a “major” criticality assessment in the report. Other deficiency types also may be includes as “minor” deficiencies in either case.

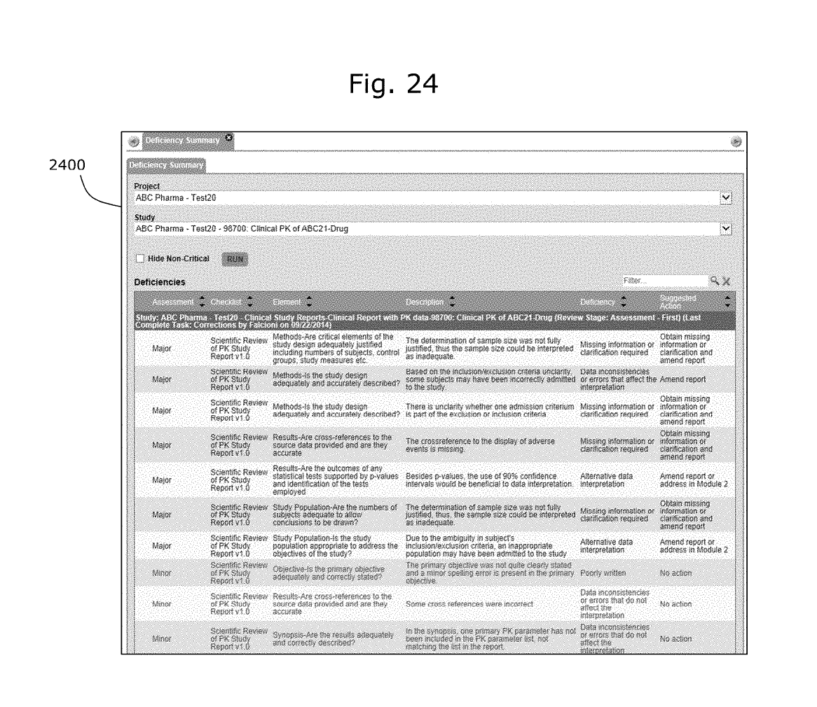

FIG. 24 shows an example of a report 2400 for a clinical study that is not intended to support the efficacy indication. In this example, the report 2400 is for a “Clinical report with PK data” study type, using a “Scientific Review of PK Study Report” checklist. Because the study is not intended to support the efficacy indication, the criticality assessments are designated either as “major” or “minor.” If this report was intended to support the efficacy indication, the “major” assessments would have been “critical.”

As noted above, the Deficiency Summary report also may include suggested actions. The suggested actions preferably are based on the deficiency type. For example, any time the reviewer selects the deficiency type “Missing information or clarification required,” the system may provide a suggestion action of “Obtain missing information or clarification and amend report.” If desired, the report may also or alternatively include suggested actions that are selected or typed by the reviewers.

The system also may be configured to allow multiple review stages for each project, study or checklist. For example, after the Final Review task has been performed for all of the checklists in a particular study, the administrator may use the Administrative Functions application (FIG. 4) to close or lock the first review stage of the study, and start the second review stage. This feature allows the system to track the typical life cycle of drug development programs, in which later stages may address deficiencies of earlier stages or additional studies may be provided over time. The use of discrete stages also allows administrators and project managers to easily and quickly review the progress of addressing and correcting deficiencies. Each review stage may include the 1s^{t} Review, Quality Control, Corrections, and Final Review tasks, as explained above; alternatively, different stages may include different tasks.

The system may provide an interface to allow users to review of all of the studies to see what stage each study is in. For example, the system may present a list of all studies associated with one or more projects (e.g., all of the projects for a particular sponsor) with an indication of the review stage for each study. Such an interface is expected to help administrators better manage the overall progress of the project, and may be helpful to make decisions about how to allocate funds and personnel resources to best advance the sponsor's overall product development goals. Other uses will be readily apparent to persons of ordinary skill in the art in view of the present disclosure.

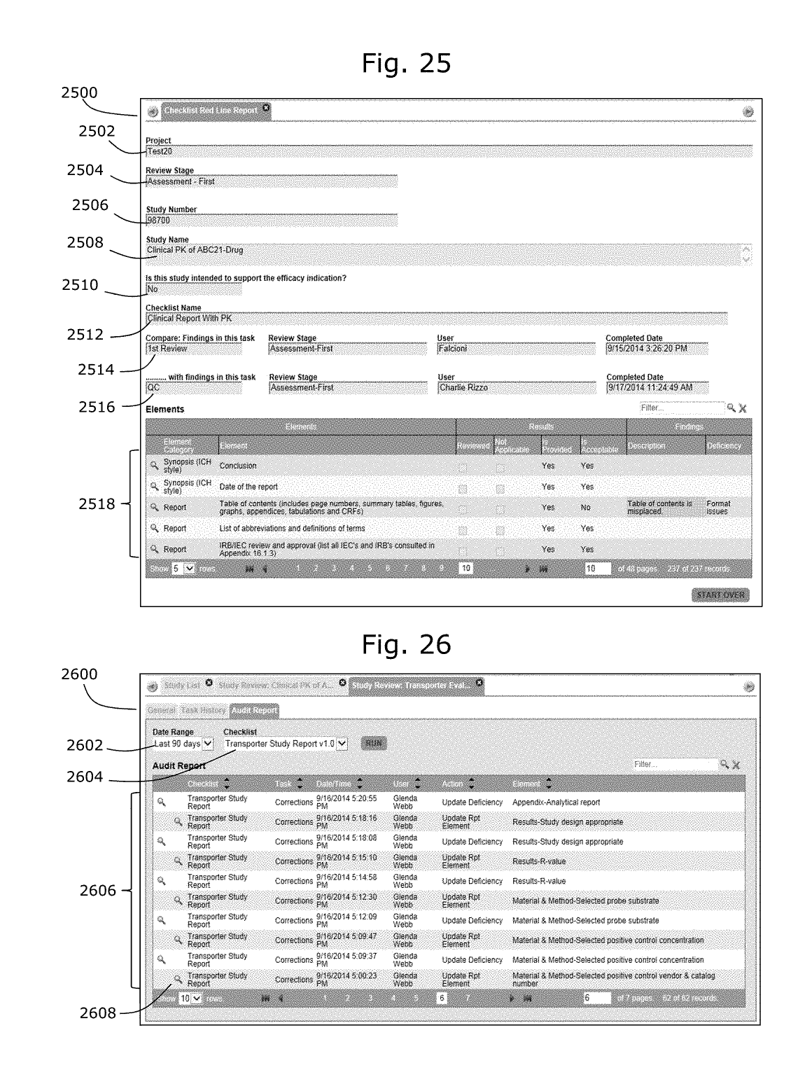

In some embodiments, the system may be configured to generate redline reports for checklists. For example, an administrator may open a Checklist Redline Reports application as shown in the interface of FIG. 4. The checklist redline report may be generated by comparing two different versions of a checklist and identifying any changes that were made between the versions. An example of a checklist redline report 2500 is shown in FIG. 25. This report 2500 identifies the project 2502, review stage 2504, study number 2506, study name 2508, criticality question indicator 2510, and checklist name 2512. The report 2500 also identifies the first version of the checklist 2514 and the second version of the checklist 2516 that are being compared. Each version may be identified by the review task (e.g., 1^{st} Review, Quality Control review, etc.), the review stage (e.g., first assessment stage, second assessment stage, etc.), the user, the completion date, or other information. The report 2500 provides a list (which may be sortable and searchable) of elements 2518 for the checklist, and indicates which elements 2518 are different. For example, the shown report 2500 highlights the “Description” of the “Table of contents . . . ” element to indicate there is a difference in the entry between the 1^{st} Review and the Quality Control review. Selecting the element presents a new interface that shows exactly what change was made.

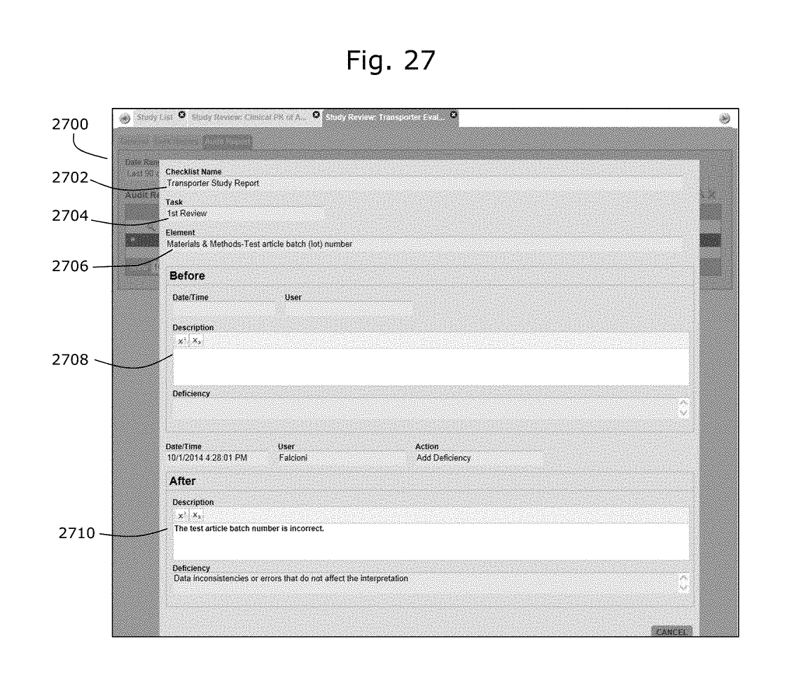

Embodiments also may be configured to generate audit reports to track the use of the system. For example, a user may be able to select a date range and one or more checklists (or projects, studies or elements), and the system will perform an audit report to show a summary of all changes to the checklist during the date range. For example, FIG. 26 shows an audit report 2600 of the “Transporter Study Report v.1.0” checklist for the last 90 days. This report 2600 indicates the date range 2602 and the selected checklist 2604, and provides a list of activities 2606 that have occurred on the checklist. Each activity 2606 may be identified by fields such as the checklist name (first column), the task (second column), the date and time of the activity (third column), the user (fourth column), the action (fifth column), and the element (sixth column). As with other interfaces that provide lists of information, the list of activities 2606 preferably is sortable and searchable.

Each activity 2606 in the list also may include an icon 2608 that may be selected to open the activity 2606 to view further details of the activity 2606. For example, selecting an activity may open an interface 2700 such as shown in FIG. 27. The audit detail interface 2700 identifies the checklist name 2702, the task 2704, the element 2706, the previous version of the element's data entry 2708, and the changed version of the element's data entry 2710. In this example, the previous version of the data entry 2708 is blank, indicating that the element had not been previously addressed before being changed as shown in the changed version of the data entry 2710. Other features and details also may be provided in the audit detail interface 2700, as will be appreciated by persons of ordinary skill in the art.

The system preferably also allows users to generate printed or electronic reports, such as reports of all of the reviewed elements, or Deficiency Summaries. In some embodiments, the system may be formatted to prepare reports that may be submitted to regulatory authorities in paper or electronic form. For example, where a private sponsor and a regulatory authority both operate versions of the system, the sponsor may electronically transmit reports to the regulatory authority as documentation of diligence and results in analyzing the supporting information. The system also may be configured to prepare reports in particular electronic formats such as electronic common technical document (“eCTD”) formats, and to otherwise prepare reports according to conventional and accepted scientific data formats. It also may be desirable to limit which users can prepare such reports. For example, in may be desirable to only allow administrators to prepare such reports by accessing a reports module 304 as shown in FIG. 3.

It will be appreciated from the foregoing description that embodiments may be useful to assist a user in a range of activities, such as: navigating the regulatory and drug development process, managing contract research services, performing data analysis, formulating research and regulatory strategies, training and overseeing reviewers, and assessing sponsor drug development programs. The system may be used to analyze supporting information, identify the risks and/or benefits of the data, and translate the assessment into documents that address health authority requirements and potential concerns while supporting a user's or sponsor's objectives. For example, a sponsor can use the system to oversee and manage the drug development lifecycle to ensure that a submission for drug approval will be successful when it is eventually made. While embodiments have particular utility in the context of obtaining approval for new drugs and the like, the system can be configured to help users evaluate other kinds of product that is or may be subject to manufacturing requirements, SOPs, or other reviews or approval processes (e.g., dietary supplements or regulated or unregulated devices used for medical purposes). As noted above, the system also may be configured to address regulatory requirements of any desired authority or guiding organization.

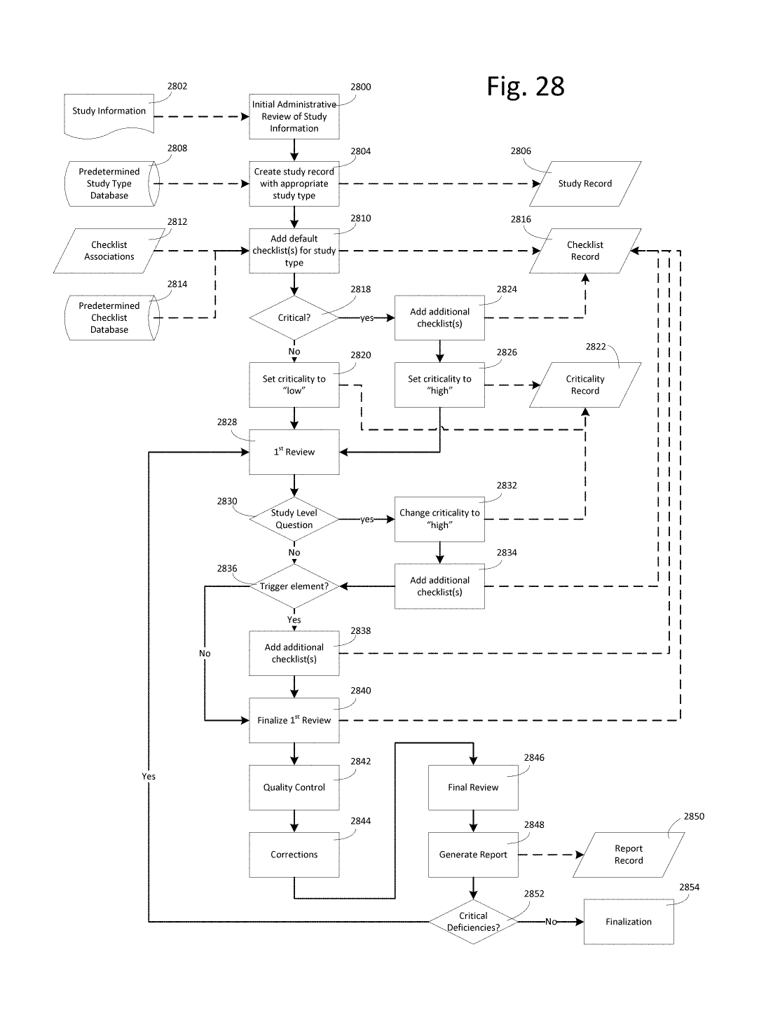

FIG. 28 illustrates a process flow that may be used in embodiments of the system, as well as examples of the manner in which certain data is accessed and saved to the system. The process flow is generally shown using solid lines, and file transactions (lookups, saves, etc.) are shown by dashed lines. The process begins at step 2800, where an administrator evaluates at least some of the study information 2802 provided, for example, by a drug sponsor. Next, in step 2804, the administrator generates a new unique study record 2806 by selecting a study type for the study information from a predetermined list of study types 2808. Upon generating the new study record, in step 2810 the system checks a predetermined association record 2812 indicating which checklists in a predetermined list of checklists 2814 are associated with the study type of the new study record 2806, and generates a new checklist record 2816 corresponding to the associated checklist(s) and the checklist's associated elements (a single checklist record 2816 containing one or more checklists is contemplated for this example, but multiple separate checklist records may be used).

In step 2818, the administrator responds to a criticality question (e.g., is this a non-clinical study that is intended to be a GLP study?, or is this a clinical study that is intended to support the efficacy indication of the drug?). If the answer is “no,” the process moves to step 2820, and the system sets the criticality of any deficiencies that might be created in the checklist record 2816 to “low,” by generating a criticality record 2822 (which may be embedded in the study record 2806, checklist record 2816, provided as a separate file, or otherwise stored) and setting a criticality variable in the criticality record 2822 to “low.” If the answer to the criticality question is “yes,” then the process moves to step 2824 and the system and/or the administrator adds one or more additional checklists (or elements) to the checklist record 2816, and then moves to step 2826 where the system generates the criticality record 2822 with the criticality variable set to “yes” to indicate that deficiencies that might be created in the checklist record 2816 may be critical.

In step 2828, the study (or one or more checklists of the study) is released and the 1^{st} Review begins. During the 1^{st} Review, the reviewer checks out the checklist, evaluates at least some of the study information 2802, and records whether each element of a checklist is reviewed, applicable, provided, or satisfactory. The recorded replies may be added to the checklist record 2816 (or an associated file) immediately, or at the end of the 1^{st} Review (step 2840) when the checklist is checked back into the system. In step 2820, the reviewer is presented with a study level query 2830, such as described above. If the answer to the study level query is “yes,” the system and/or reviewer changes the criticality record 2822 to “high” in step 2832, and in step 2834 the system and/or reviewer may add one or more additional checklists (or elements) to the checklist record 2816. At the conclusion of step 2834, or if the answer to the study level query in step 2830 is “no,” the process continues to step 2836. In step 2836, the system and/or reviewer evaluates the answer(s) to any trigger elements, such as described above, that might be included in the checklist elements. If the answer is “yes,” the process proceeds to step 2838, where the system and/or reviewer adds one or more additional checklists (or elements) to the checklist record 2816. At the end of step 2838, or if the answer to the trigger element query 2836 is “no,” the process proceeds to step 2840, where the 1^{st} Review is finalized. At this point, the checklist is checked back into the system, and any data entries for elements that have not already been saved to the checklist record are saved there.

The process then proceeds to Quality Control in step 2842, Corrections in step 2844, and Final Review in step 2846. Each of these steps may include additional process steps similar to those that are conducted between the start and finalization of the 1^{st} review (e.g., Quality Control may include answering study level queries, and evaluating trigger elements, changing criticality and adding checklists or elements). Other steps, such as preparing reports, performing checklist redlines, and the like, also may be added throughout the process.

After the Final Review step 2846 is complete, a user or the system may generate a report in step 2848 and save the report to a report record 2850 associated with the checklist. The report may be indicate whether the study information is not adequate for any of the repot elements. For example, the report may be a deficiency summary report, such as described above, indicating whether any elements are deficient, and suggesting remedial action. In step 2852, the system or a user evaluates whether any element includes a critical deficiency. If there are any critical deficiencies, the process may start again at the 1^{st} Review step 2828 to commence an additional review stage, in which any deficiencies may be addressed and corrected. If there are no critical deficiencies, then the process may move to finalization for submission to the relevant regulatory authority in step 2854.

The additional checklists (or elements) selected in steps 2824, 2834 and 2838 may be chosen based on additional checklist associations provided in the checklist association record 2812, or selected using other logical processes. Furthermore, some steps of the process may be automated, while others are performed manually. For example, in preferred embodiments, one or both of the steps of changing the criticality record 2822 to “high” (steps 2826 and 2832) preferably are automatically performed upon answering the relevant question without reviewer approval. It is also preferred for the step of adding additional checklists in reply to a trigger element (step 2838) to be automated and performed without reviewer approval. As another example, it is preferred for step 2834 to be at least partly manually performed by the reviewer to provide an opportunity for the reviewer to determine whether the added checklists are redundant or unnecessary. Other alternatives will be readily apparent to persons of ordinary skill in the art in view of the present disclosure.

For organizational purposes, each of the records may be stored in a common system database, and may include mirrored files in separate databases to provided redundant storage. Each record may include a variable to associate the record with other records. For example, each record may include a study name variable that associates each record with a particular study, and may include other variables to make other associations. Other types of storage and association regimes may be used instead, and the manners in which the records are stored and associated are not critical to the embodiments.

It will be appreciated that the process of FIG. 28 is described as operating in one example of many possible orders. The process may be readily modified by reversing steps (e.g., reversing the order of steps 2824 and 2826, steps 2832 and 2834, or steps 2830 and 2836), combining steps, adding new steps, and removing steps. The decision steps also may be modified by reversing the logic of the answer (e.g., the critical question in step 2818 may be whether the study is a non-clinical study that is intended NOT to be a GLP study, in which case the “yes” and “no” flow paths would be reversed). It will also be appreciated that multiple instances of the process may be operated at the same time by one or more system users, and each instance of the process may be at a different processing step or stage. Other alternatives will be readily apparent to persons of ordinary skill in the art in view of the present disclosure.

The process described in FIG. 28, and other implementations of embodiments of systems or features of the invention, may be implemented using computer readable instructions configured to cause a computing device or the like to perform the process steps or provided the features and capabilities described herein. For example, the instructions may be stored as a program in a computing device's memory, and accessed and executed using one or more computing device processors. Such instructions may be provided in the form of software, hardware, or the like. Some or all of the instructions may be downloadable or installed locally on a computing device, hosted and accessed remotely via a wireless or wired network (e.g., the Internet), or combinations thereof.

In one example, the list of study types 2808, list of checklists 2814, and the checklist association record 2812 may be stored as one or more files in the memory of one or more computers, such as a server system comprising one or more servers 115, 117₁, 117ₙ, etc. These files also may be stored on client computers 105, 107₁, 107ₙ, etc. As used herein, the term “file” is intended to cover any kind of data storage regime, including separate data records (e.g., the list of study types 2808 is a completely different data record as the list of checklists 2814), collective data records (e.g., the list of study types 2808 is stored collectively with the list of checklists 2814 in a database structure), and so on. It will also be appreciated that files can be distributed among memories in different servers and other computers. Other alternatives will be readily apparent to persons of ordinary skill in the art in view of the present disclosure.

The headings used herein are for organizational purposes only and are not meant to limit the scope of the description or the claims. As used throughout this application, the word “may” is used in a permissive sense (i.e., meaning having the potential to), rather than the mandatory sense (i.e., meaning must). Similarly, the words “include,” “including,” and “includes” mean including but not limited to. Furthermore, the fact that some embodiments or features are described as exemplary, optional or the like is not intended to suggest that features not so described are mandatory. Also, the terms “or” and “and” are used herein in a nonexclusive manner, and are intended to cover both conjunctive and alternative selections of items in a list (i.e., “and” means “and/or” and “or” means “and/or”).

In the foregoing detailed description, numerous specific details are set forth in order to provide a thorough understanding of exemplary embodiments or other examples described herein. However, it will be understood that these examples may be practiced without the specific details, or with the details in alternative arrangements (e.g., moving the prompt to answer a criticality question into a separate interface, subdividing interfaces, or providing alternative menu architectures). In other instances, well-known methods, procedures, components and circuits have not been described in detail, so as to not obscure the description. Further, the examples disclosed herein are for exemplary purposes only and other examples may be employed in lieu of, or in combination with, the examples disclosed. It should also be noted that the examples presented herein should not be construed as limiting of the scope of embodiments of the present disclosure, as other equally effective examples are possible and likely.

It will also be understood that, while the foregoing is directed to exemplary embodiments of the present disclosure, other and further embodiments of the systems described herein may be devised without departing from the basic scope of the disclosure, and should be considered part of this disclosure, as if described fully herein. For example, whereas the worldwide web and mobile web are growing content and capabilities at ever-increasing rates, the ability to adapt the systems, methods, applications, and interfaces disclosed herein to existing or new mobile- or web-based technology is contemplated by embodiments of the present disclosure and such modifications would not depart the scope of the disclosure disclosed herein.

1-27. (canceled)

28. A method for optimizing data entry in a computer-implemented drug study program, the method comprising executing with a processor the steps of:
uniquely identifying a drug study;
assigning a predetermined study type to the drug study, the study type being selected from a plurality of predetermined study types maintained in a memory accessible by the processor;
identifying the drug study as being a nonclinical study associated with laboratory practice guidelines, a clinical study relating to an efficacy indication of a drug, or another type of study;
dynamically selecting at least one checklist from a plurality of different checklists based on the status of the drug study as a nonclinical study or a clinical study, the at least one checklist comprising a plurality of elements each comprising an identification of one or more features to be identified in the drug study, and one or more queries associated with the respective one or more features including at least one query representing a deficiency type selected from a predetermined list of deficiency types;
wherein the dynamic selection of the at least one checklist comprises identifying one or more elements or queries relevant to the study type and including them for presentation to a user of the program, and identifying one or more elements or queries not relevant to the study type and excluding them from presentation to the user of the program, to thereby reduce irrelevant information presentation to the program user and prevent accumulation of excess data entries being stored by the program.

29. The method of claim 28, further comprising:
presenting the plurality of elements to the at least one user interface;
receiving a respective reply to each of the plurality of elements; and
assigning a respective criticality value to each respective reply comprising a selection of a respective deficiency type from the predetermined list of deficiency types by:
assigning a high respective criticality value if the drug study is a nonclinical study associated with laboratory practice guidelines or a clinical study relating to an efficacy indication of a drug, or
assigning a moderate respective criticality value if the drug study another type of study.

30. The method of claim 28, further comprising:
one or more additional elements to the plurality of elements based on the study type.

31. The method of claim 30, wherein the one or more additional elements comprises aspects of (i) a bioanalytical method validation report, (ii) an analytical method validation report, or (iii) a biological sample analysis report.

32. The method of claim 28, further comprising:
receiving a selection of at least one additional checklist from a program user.

33. The method of claim 28, wherein the predetermined list of deficiency types comprises a high risk group of deficiency types representing deficiencies that affect the acceptability of the drug study information and a low risk group of deficiency types representing deficiencies that do not affect the acceptability of the drug study information.

34. The method of claim 28, wherein at least one of the plurality of elements comprises a trigger element comprising a question regarding the content of the drug study, and an associated dynamic checklist comprising one or more additional elements regarding the drug study; and
wherein the method further comprises adding the one or more additional elements to the plurality of elements upon receiving, from a user interface, a predetermined reply to the question regarding the content of the drug study information.

35. The method of claim 34, wherein the question regarding the content of the drug study information comprises a question whether the drug study information includes kinetic or blood level data.

36. The method of claim 34, wherein the question regarding the content of the drug study information comprises a question whether the drug study information includes metabolism and transporter drug interaction-related data.

37. The method of claim 34, wherein the question regarding the content of the drug study information comprises a question whether the drug study information includes toxicology-related data.

38. The method of claim 34, wherein the step of adding the one or more additional elements to the plurality of elements comprises:
presenting a list of one or more additional checklists to a user terminal;
receiving a selection of at least one of the one or more additional checklists from the user terminal; and
associating the selected at least one additional checklist with the drug study.

39. The method of claim 38, wherein the step of presenting a list of one or more additional checklists to the at least one user terminal comprises presenting a list of additional drug studies to which the one or more additional checklists were previously associated.

40. The method of claim 28, wherein the one or more queries further comprises:
a review status indicator to indicate whether the drug study information has been evaluated to respond to the respective element;
an applicability indicator to indicate whether the element is applicable to the drug study; and
an acceptability indicator to indicate whether the drug study element contains the one or more features to the be identified in the drug study.

41. The method of claim 28, wherein at least one of the elements comprises one or more regulatory sources for information regarding one or more features to be identified in the drug study.

42. The method of claim 28, wherein at least one of the deficiency types from the predetermined list of deficiency types comprises an associated record indicating a corrective action to take to overcome the deficiency type.
